(19) United States
(12) Patent Application Publication
(10) Pub. No.: US 2009/0086140 A1
(43) Pub. Date: Apr. 2, 2009

(54) PHOTO ALIGNMENT FILM AND RETARDATION FILM, AND THEIR APPLICATIONS, AND COMPOSITIONS AND METHODS FOR PRODUCING THEM

(75) Inventors: Takayasu Yasuda, Minami-ashigara-shi (JP); Michitaka Matsuumi, Minami-ashigara-shi (JP); Shinichi Morishima, Minami-ashigara-shi (JP)

Correspondence Address: BUCHANAN, INGERSOLL & ROONEY PC, POST OFFICE BOX 1404, ALEXANDRIA, VA, 22313-1404, US

(73) Assignee: FUJIFILM Corporation, Minato-ku (JP)

(21) Appl. No.: 12/239,246

(22) Filed: Sep. 26, 2008

(30) Foreign Application Priority Data
Sep. 28, 2007 (JP) 2007-255231

(51) Int. Cl. G02F 1/1337; C08L 79/00; C08L 27/12; B05D 3/06; C07C 69/767

(52) U.S. Cl. 349/123; 525/328.4; 525/326.2; 427/487; 560/60

(57) ABSTRACT

Disclosed is a composition comprising at least one polymer compound represented by formula (1) below, and at least one polymerizable compound.

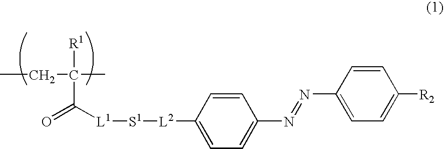

### CROSS-REFERENCE TO RELATED APPLICATIONS

This application claims benefit of priority under 35 U.S.C. 119 to Japanese Patent Application No. 2007-255231 filed on Sep. 28, 2007; and the entire contents of the application are incorporated herein by reference.

### BACKGROUND OF THE INVENTION

1. Field of the Invention

The present invention relates to a photo-alignment film for aligning liquid crystal, retardation film, a composition useful for producing the same, and a method of producing the same. The present invention relates also to a liquid crystal cell comprising the photo-alignment film and a liquid crystal display device comprising the liquid crystal cell or the retardation film.

2. Related Art

There are known methods of chemically or physically treat the surface of a substrate, as a method for aligning liquid crystal on the substrate. One method, for aligning liquid crystal in a homogenous alignment state in which the liquid crystal is aligned uniformly and unidirectionally, comprises forming a polymer film typically formed of polyimide on the surface of a support, and then rubbing the surface of the film unidirectionally with cloth or the like. The method has been used for preparing an alignment film for liquid crystal display device. The method, however, suffers from a problem in that the yield ratio may degrade due to static electricity and dust in the process of rubbing, and due to difficulty in quantitative control of alignment.

Photo-assisted alignment has attracted a good deal of attention as a method of solving these problems arisen from the rubbing. One example is a method employing photo-isomerization of azobenzene derivatives for controlling alignment (Japanese Patent No. 2990270). More specifically, according to the example, an alignment layer, a compound layer capable of causing photo-isomerization reaction is formed on the surface of a support, and irradiated with light, thereby being capable of controlling alignment.

On the other hand, there have been proposed a various modes of liquid crystal display devices. Among those, VA (vertically aligned) mode display has been proven to be a wide viewing angle mode display capable of omni-directionally achieving desirable contrast viewing-angle characteristics, and has already been disseminated into families as being applied to television sets. Large-size displays of 30 inches or larger have been launched. In the VA mode liquid crystal display device, optically anisotropic film or the like, having various characteristics, have been used for optical compensation, for the purpose of reducing leakage of light and color shift observed in oblique directions in the black state.

For example, a retardation plate satisfying predetermined optical characteristics has been proposed, as an optically compensation sheet contributive to improvement in color-viewing angle characteristics of VA-mode liquid crystal display devices, wherein modified polycarbonate has been used as the material therefor (JPA No. 2004-37837).

There has been proposed also systems of independently compensating three colors of R, G and B (GB2394718, and JPA Nos. 2004-240102, 2005-4124, 2005-24919, 2005-24920 and 2006-78647). According to one example, a retardation layer is formed in a liquid crystal cell, and is subjected to a patterning treatment together with a color filter and so forth. The patterning treatment of the retardation layer formed in the liquid crystal cell, however, needs complicated operations including, for example, forming an alignment layer in the cell, rubbing the alignment layer, applying a polymerizable liquid crystal composition to the rubbed surface, followed by alignment and fixation to thereby form the retardation layer, further forming a resist layer for patterning the retardation layer, etching the retardation layer, and removing the resist layer. It is, therefore, difficult to form the patterned retardation layer, having optically uniform retardation characteristics, in the liquid crystal cell. Another problem is that the retardation film is exposed to heat and photoresist solvent in the process of resist patterning; thereby the retardation film may be occasionally altered in the retardation before and after the etching.

On the other hand, as a material for the retardation film, there has been proposed a birefringence-inducing material. One example of such a material is a composition containing naphthyl acryloyl or its derivatives or biphenyl acryloyl or its derivatives; and birefringence is induced due to molecular motion and subsequent molecular orientation generated by irradiating the composition with light or heat (JPA Nos. 2004-258426 and 2006-308878).

### SUMMARY OF THE INVENTION

The conventional photo-alignment films have, however, been suffering from a problem of poor durability against light or the like.

It is, therefore, an object of the present invention to provide a photo-alignment film excellent in durability typically against light, a composition for photo-alignment film useful for producing the same, and a method of producing the same.

It is another object of the present invention to provide a liquid crystal cell and a liquid crystal display device having the photo-alignment film described in the above.

The present inventors further found out from the investigations that the materials proposed in JPA Nos. 2004-258426 and 2006-308878 may sometimes fail in obtaining desired retardation necessary for optical compensation, and may further raise a problem in that the retardation may vary due to various treatments such as heating, solvent treatment and so forth carried out in the process of producing the liquid crystal cell.

It is, therefore, still another object of the present invention to provide a novel retardation film useful for optical compensation of liquid crystal display devices, a method and a composition useful for producing the same.

The means for achieving the above mentioned objects are as follows.
[1] A composition for photo-alignment film comprising at least one polymer compound represented by formula (1) below, and at least one polymerizable compound:

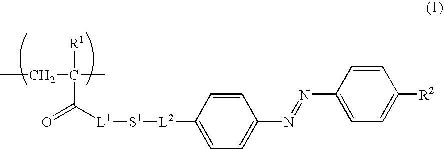

where, R¹ represents a hydrogen atom or methyl group; L¹ represents —O—, —NR³ — (R³ represents a hydrogen atom or methyl group) or —S—; S¹ represents a divalent linking group selected from the group consisting of a single bond, —O—, —S—, —C(═O)—, —SO₂—, —NH—, —CH₂—, —CH═CH—, —C≡C— and combinations thereof, wherein any of which having hydrogen atom(s) may have substituent (s) in place of the hydrogen atom(s); L² represents a single bond, —O—, —NR⁴— (R⁴ represents a hydrogen atom or methyl group), —S—, —OCO₂—, —CO₂— or —OCO—; R² represents a hydrogen atom, non-substituted or substituted alkylene group, —CN, —NO₂, non-substituted or substituted alkoxy group, —F, —Br, —Cl, —CF₃, —CO₂R⁵ (R⁵ represents a non-substituted or substituted alkyl group), —≡—R⁶ (R⁶ represents a hydrogen atom or non-substituted or substituted alkyl group) or

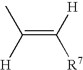

where R⁷ represents a hydrogen atom, or substituted or non-substituted alkyl group.
[2] The composition for photo-alignment film as set forth in [1], wherein said polymerizable compound is a compound having two or more polymerizable groups.
[3] The composition for photo-alignment film as set forth in [1] or [2], comprising two or more polymerizable compounds, at least one of which being a compound having one polymerizable group, and at least other of which being a compound having two or more polymerizable groups.
[4] The composition for photo-alignment film as set forth in any one of [1] to [3], wherein said at least one polymerizable compound is a compound represented by formula (2), (3) or (4):

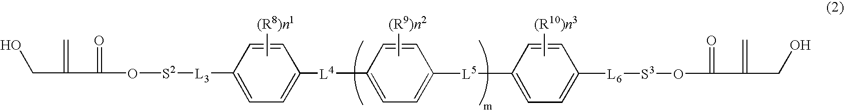

where each of R⁸, R⁹ and R¹⁰ independently represents —F, —Br, —Cl, —CH₃ or —OCH₃ and is same or different at each occurrence; each of n¹ to n³ independently represents an integer of 0 to 4; each of S² and S³ independently represents a divalent linking group selected from the group consisting of a single bond, —O—, —S—, —C (═O)—, —SO₂—, —NH—, —CH₂—, —CH═CH— and —C≡C—, and combinations of them, wherein any of which containing hydrogen atom(s) may have substituent(s) in place of the hydrogen atom(s); each of L³ and L⁶ independently represents a single bond, —O—, —S—, —NR¹¹— (R¹¹ represents a hydrogen atom of methyl group), —CO₂—, —OCO₂— or —OCO—; each of L⁴ and L⁵ independently represents a single bond, —CO₂—, —OCO—, —CONR¹²— (R¹² represents a hydrogen atom or methyl group) —NR¹³CO— (R¹³ represents a hydrogen atom or methyl group), —O—, —S—, —C(CH₃)₂—, —≡— or

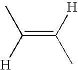

where m represents an integer of 0 to 3;

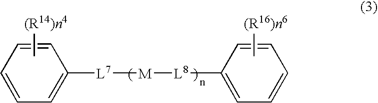

where each of R¹⁴ and R¹⁶ independently represents Q-L⁹-S⁴-L¹⁰-, where Q represents a hydrogen atom or polymerizable group, each of L⁹ and L¹⁰ independently represents a single bond, —O—, —S—, —NR¹⁷— (R¹⁷ represents a hydrogen atom or methyl group), —CO₂—, —OCO₂— or —OCO—, and S⁴ represents a divalent linking group selected from the group consisting of a single bond, —O—, —S—, —C(═O)—, —SO₂—, —NH—, —CH₂—, —CH═CH—, —C≡C— and combinations of them, wherein any of which having hydrogen atom(s) may have substituent(s) in place of the hydrogen atom(s); each of n⁴ and n⁶ independently represents an integer of 0 to 5 and the sum of n⁴ and n⁶ is from 1 to 10, n⁴ “R¹⁴”s and n⁶ “R¹⁶”s may independently differ from each other, at least one of n⁴ “R¹⁴”s and n⁶ “R¹⁶”s has a polymerizable group Q; M represents the group shown below:

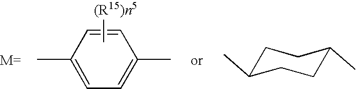

where R¹⁵ represents —F, —Br, —Cl, —CH₃ or —OCH₃, n⁵ represents an integer of 0 to 4, and n⁵ “R¹⁵”s may differ from each other; each of L⁷ and L⁸ independently represents a single bond, —CO₂—, —OCO—, —CONR¹⁷— (R¹⁷ represents a hydrogen atom or methyl group), —NR¹⁸CO— (R¹⁸ represents a hydrogen atom or methyl group), —O—, —S—, —C(CH₃)₂—, —≡— or

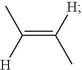

and n represents an integer from 0 to 3; provided that formula (3) has at least one polymerizable group;

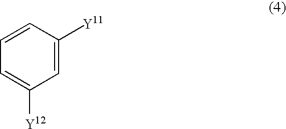

where each of Y¹¹ and Y¹² independently represents formula (4-A), formula (4-B) or formula (4-C) below, provided that formula (4) has at least one polymerizable group:

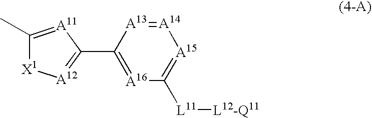

where A¹¹, A¹², A¹³, A¹⁴, A¹⁵ and A¹⁶ each represent a methine or nitrogen atom; X¹ represents an oxygen atom, sulfur atom, methylene or imino; L¹¹ represents —O—, —O—CO—, —CO—O—, —O—CO—O—, —S—, —NH—, —SO₂—, —CH₂—, —CH═CH— or —C≡C—; L¹² represents a divalent linking group selected from the group consisting of —O—, —S—, —C(═O)—, —SO₂—, —NH—, —CH₂—, —CH═CH—, —C≡C— and combinations thereof wherein any of which having hydrogen atom(s) may have substituent (s) in place of the hydrogen atom(s); and Q¹¹ represents a polymerizable group or hydrogen atom;

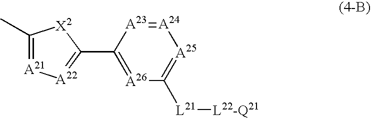

where A²¹, A²², A²³, A²⁴, A²⁵ and A²⁶ each independently represent a methine or nitrogen atom; X² represents an oxygen atom, sulfur atom, methylene or imino; L²¹ represents —O—, —O—CO—, —CO—O—, —O—CO—O—, —S—, —NH—, —SO₂—, —CH₂—, —CH═CH— or —C≡C—; L²² represents a divalent linking group selected from the group consisting of —O—, —S—, —C(═O)—, —SO₂—, —NH—, —CH₂—, —CH═CH—, —C≡C— and combinations thereof wherein any of which having hydrogen atom(s) may have substituent (s) in place of the hydrogen atom(s); and Q²¹ represents a polymerizable group or hydrogen atom;

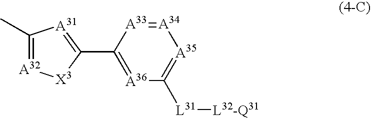

where A³¹, A³², A³³, A³⁴, A³⁵ and A³⁶ each independently represents a methine or nitrogen atom; X³ represents an oxygen atom, sulfur atom, methylene or imino; L³¹ represents —O—, —O—CO—, —CO—O—, —O—CO—O—, —S—, —NH—, —SO₂—, —CH₂—, —CH═CH— or —C≡C—; L³² represents a divalent linking group selected from the group consisting of —O—, —S—, —C(═O)—, —SO₂—, —NH—, —CH₂—, —CH═CH—, —C≡C— and combinations thereof wherein any of which having hydrogen atom(s) may have substituent(s) in place of the hydrogen atom(s); and Q³¹ represents a polymerizable group or hydrogen atom.
[5] The composition for photo-alignment film as set forth in any one of [1] to [4], further comprising a polymerization initiator.
[6] A composition for retardation film comprising at least one polymer compound represented by formula (1) as set forth in [1] and at least one polymerizable compound represented by formula (2), (3) or (4) as set forth in [4].
[7] The composition for retardation film as set forth in [6], further comprising a polymerization initiator.
[8] A photo-alignment film formed of a composition as set forth in any one of [1] to [5].
[9] A photo-alignment film formed of a composition as set forth in any one of [1] to [5] irradiated with light.
[10] A retardation film formed of a composition as set forth in [6] or [7].
[11] A retardation film formed of a composition as set forth in [6] or [7] irradiated with light.
[12] A liquid crystal cell comprising a pair of substrates and a liquid crystal composition held therebetween, wherein at least one of said pair of substrates has a photo-alignment film as set forth in [8] or [9] on the inner surface thereof.
[13] A liquid crystal display device comprising a liquid crystal cell as set forth in [12].
[14] The liquid crystal display device as set forth in [13], employing an IPS-mode or TN-mode.
[15] A liquid crystal display device comprising a retardation film as set forth in [10] or [11].
[16] A method of producing a photo-alignment film comprising applying a composition as set forth in any one of [1] to [5] to a surface to form a layer of the composition, and irradiating the layer with polarized light or irradiating with non-polarized light in an oblique direction.
[17] A method of producing a retardation film comprising applying a composition as set forth in [6] or [7] to a surface to form a layer of the composition, and irradiating the layer with polarized light or irradiating with non-polarized light in an oblique direction.
[18] The method as set forth in [16] or [17], further comprising heating the layer of the composition following the irradiating.
[19] The method as set forth in [18], wherein the heating is carried out at 50° C. to 240° C.
[20] The method as set forth in any one of [16] to [19], further comprising irradiating the layer with non-polarized light in normal line direction relative to the layer plane, following the irradiating with polarized light or irradiating with non-polarized light in an oblique direction.
[21] A liquid crystal compound represented by formula (2):

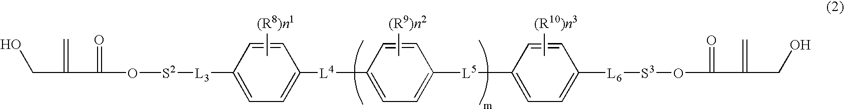

where each of R⁸, R⁹and R¹⁰ independently represents —F, —Br, —Cl, —CH₃ or —OCH₃ and is same or different at each occurrence; each of n¹ to n³ independently represents an integer of 0 to 4; each of S² and S³ independently represents a divalent linking group selected from the group consisting of a single bond, —O—, —S—, —C(═O)—, —SO₂—, —NH—, —CH₂—, —CH═CH— and —C≡C—, and combinations of them, wherein any of which containing hydrogen atom(s) may have substituent(s) in place of the hydrogen atom(s); each of L³ and L⁶ independently represents a single bond, —O—, —S—, —NR¹¹— (R¹¹ represents a hydrogen atom of methyl group), —CO₂—, —OCO₂— or —OCO—; each of L⁴ and L⁵ independently represents a single bond, —CO₂—, —OCO—, —CONR¹²— (R¹² represents a hydrogen atom or methyl group), —NR¹³CO— (R¹³ represents a hydrogen atom or methyl group) , —O—, —S—, —C(CH₃)₂—, —≡— or

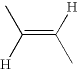

where m represents an integer of 0 to 3.

### DETAILED DESCRIPTION OF THE INVENTION

The invention will be described in detail below. The expression “from a lower value to an upper value” referred herein means that the range intended by the expression includes both the lower value and the upper value.

### [Composition for Photo-Alignment Film]

The present invention relates to a composition for photo-alignment film comprising at least one polymer compound represented by formula (1), and at least one polymerizable compound.

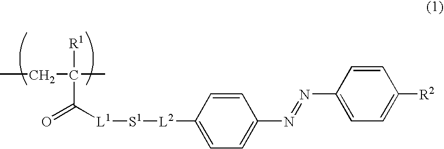

In formula (1), R¹ represents a hydrogen atom or methyl group.

In formula (1), L¹ represents —O—, —NR³— (R³ represents a hydrogen atom or methyl group) or —S—, wherein —O— and —NR³— (R³ represents a hydrogen atom or methyl group) are preferable, and —O— is still more preferable.

In formula (1), S¹ represents a divalent linking group selected from the group consisting of a single bond, —O—, —S—, —C(═O)—, —SO₂—, —NH—, —CH₂—, —CH═CH—, —C≡C— and combinations thereof; and, preferably, S¹ contains 0 to 16 units of —CH₂— and has 0 to 20 carbon atoms, more preferably, contains 0 to 11 units of —CH₂— and has 0 to 15 atoms, and even more preferably contains 0 to 8 units of —CH₂— and has 0 to 10 carbon atoms. More preferably, S¹ is selected from the group set consisting of a single bond, —O—, —C(═O)—, CH₂— and combinations thereof, and has 0 to 10 carbon atoms. Hydrogen atom(s) of —NH—, —CH₂—, —CH═CH— may be replaced by substituent(s). Preferable examples of this sort of substituent include halogen atoms, cyano, nitro, alkyl group having 1 to 6 carbon atoms, halogen atom-substituted alkyl group having 1 to 6 carbon atoms, alkoxy group having 1 to 6 carbon atoms, acyl group having 2 to 6 carbon atoms, alkylthio group having 1 to 6 carbon atoms, acyloxy group having 2 to 6 carbon atoms, alkoxycarbonyl group having 2 to 6 carbon atoms, carbamoyl group, carbamoyl group with alkyl group having 2 to 6 carbon atoms, and acylamino group having 2 to 6 carbon atoms, wherein alkoxy group having 1 to 6 carbon atoms, and alkyl group having 1 to 6 carbon atoms are more preferable.

S¹ expecially preferably represents a single bond or non-substituted polymethylene group composed of 2 to 6 units of —CH₂—.

In formula (1), L² represents a single bond, —O—, —NR⁴— (R⁴ represents a hydrogen atom or methyl group), —S—, —OCO₂—, —CO₂— or —OCO—; wherein single bond, —O—, —OCO₂— and CO₂— are preferable, and a single bond and —O— are more preferable.

In formula (1), R² represents a hydrogen atom, non-substituted or substituted alkylene group, —CN, —NO₂, non-substituted or substituted alkoxy group, —F, —Br, —Cl, —CF₃, —CO₂R⁵ (R⁵ represents a non-substituted or substituted alkyl group), —≡—R⁶ (R⁶ represents a hydrogen atom or non-substituted or substituted alkyl group) or

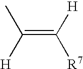

where R⁷ represents a hydrogen atom, or substituted or non-substituted alkyl group.

In formula (2), R² is preferably a hydrogen atom, —CN, —NO₂, substituted alkoxy group, —F, —Br, —Cl, —CF₃, —CO₂R⁵ (R⁵ represents a non-substituted or substituted alkyl group), —≡—R⁶ (R⁶ represents a hydrogen atom or non-substituted or substituted alkyl group) or

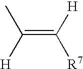

where R⁷ represents a hydrogen atom, or substituted or non-substituted alkyl group;
and still more preferably —CN, —F or substituted alkoxy group. The number of carbon atoms of non-substituted or substituted alkylene group represented by R², non-substituted or substituted alkoxy group represented by R², non-substituted or substituted alkyl group represented by R⁵, R⁶ and R⁷ is preferably 1 to 20, more preferably 1 to 15, and still more preferably 1 to 8. Examples of substituted alkylene group represented by R², substituted alkoxy group represented by R², and substituted alkyl group represented by R⁵, R⁶ and R⁷ include the substituents exemplified by those for S¹, and polymerizable group. Examples of the polymerizable group include:

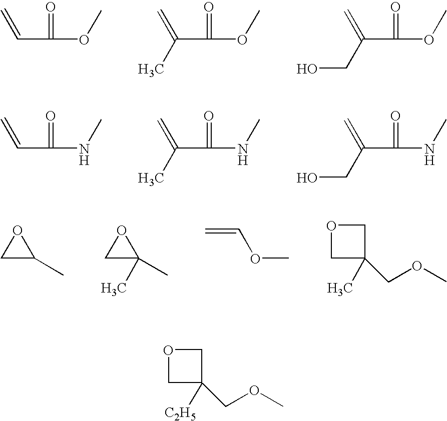

among which, more preferable examples include:

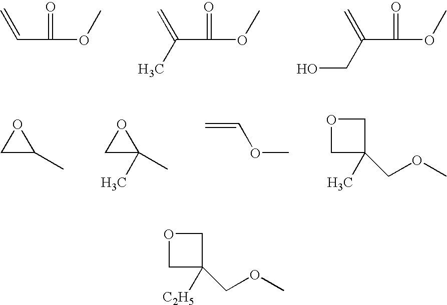

and still more preferable examples include:

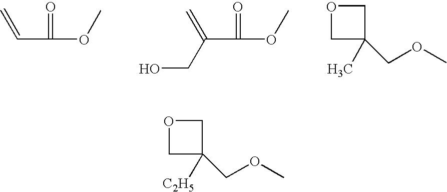

The composition for photo-alignment film of the present invention comprises at least one polymerizable compound. The polymerizable compound may be selected from polymerizable compounds having two or more polymerizable groups (referred to as “multifunctional monomer”, hereinafter) or may be selected from polymerizable compounds having only a single species of polymerizable group (referred to as “mono-functional monomer”, hereinafter), wherein the multi-functional monomer is preferable in terms of improving the durability. The multi-functional monomer may also preferably be used in combination with the mono-functional monomer.

At least one polymerizable compound is preferably selected from the compounds represented by formulas (2), (3) and (4) below. Selection of two or more species is more preferable. When two or more species are selected, they may be selected from the same formula, such as two species from formula (2), two species from (3) or two species from (4), or those selected from the different formulas, such as the one from (2) and the other from (3), may be combined.

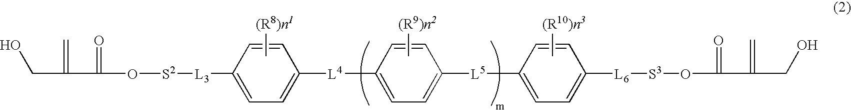

In formula (2), each of R⁸, R⁹ and R¹⁰ independently represents —F, —Br, —Cl, —CH₃ or —OCH₃, more preferably —F, —Cl, —CH₃ or —OCH₃; and still more preferably —F, —CH₃ or —OCH₃.

Each of n¹ to n³ independently represents an integer of 0 to 4, more preferably 0 to 2, and more preferably 0 or 1. In the formula, R⁸, R⁹, or R¹⁰ is same or different at each occurrence.

In the formula, each of S² and S³ independently represents a divalent linking group selected from the group consisting of a single bond, —O—, —S—, —C(═O)—, —SO₂—, —NH—, —CH₂—, —CH═CH— and —C≡C—, and combinations of them, wherein any of which containing hydrogen atom(s) may have substituent(s) in place of the hydrogen atom(s). The divalent group preferably contains 1 to 16 units of —CH₂— and 1 to 20 carbon atoms, more preferably contains 1 to 11 units of —CH₂— and 1 to 15 carbon atoms, and still more preferably contains 1 to 8 units of —CH₂— and 1 to 10 carbon atoms. Still more preferably, each of S² and S³ is independently a divalent group having 1 to 10 carbon atoms, selected from the group consisting of a single bond, —O—, —C(═O)—, CH₂— and combinations of them. Hydrogen atom(s) of —NH—, —CH₂— and —CH═CH— may be replaced by substituent(s). Examples of the substituent include halogen atoms, cyano, nitro, alkyl group having 1 to 6 carbon atoms, halogen-substituted alkyl group having 1 to 6 carbon atoms, alkoxy group having 1 to 6 carbon atoms, acyl group having 2 to 6 carbon atoms, alkylthio group having 1 to 6 carbon atoms, acyloxy group having 2 to 6 carbon atoms, alkoxycarbonyl group having 2 to 6 carbon atoms, carbamoyl group, carbamoyl with an alkyl group having 2 to 6 carbon atoms, and acylamino group having 2 to 6 carbon atoms, wherein more preferable examples include alkoxy group having 1 to 6 carbon atoms and alkyl group having 1 to 6 carbon atoms.

Especially preferably, each of S² and S³ is a divalent group independently selected from the group consisting of combinations of —O— and CH₂—, containing 1 to 8 units of non-substituted CH₂—, and 0 to 3 units of —O—.

In the formula, each of L³ and L⁶ independently represents a single bond, —O—, —S—, —NR¹¹— (R¹¹ represents a hydrogen atom or methyl group), —CO₂—, —OCO₂— or —OCO—, wherein —O—, —CO₂—, —OCO₂— or —OCO— is more preferable, and —O— or —CO₂— is especially preferable.

In the formula, each of L⁴ and L⁵ independently represents a single bond, —CO₂—, —OCO—, —CONR¹²— (R¹² represents a hydrogen atom or methyl group), —NR¹³CO— (R¹³ represents a hydrogen atom or methyl group), —O—, —S—, —C(CH₃)₂—, —≡— or

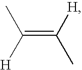

more preferably a single bond, —CO₂—, —OCO—, —CONR¹²— (R¹² represents a hydrogen atom or methyl group), —NR¹³CO— (R¹³ represents a hydrogen atom or methyl group) or —≡—, and still more preferably a single bond, —CO₂— or —OCO—.

In the formula, m represents an integer of 0 to 3, and more preferably 0 to 2.

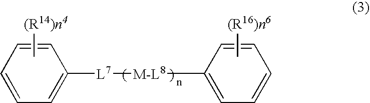

In formula (3), each of R¹⁴ and R¹⁶ independently represent Q-L⁹-S⁴-L¹⁰ -, where Q represents a hydrogen atom or polymerizable group, wherein preferable examples of the polymerizable group include:

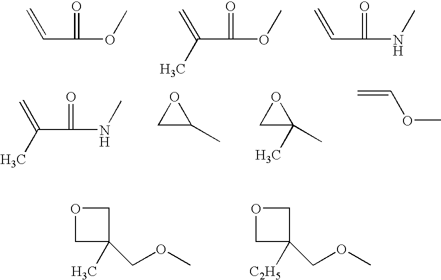

more preferable examples include:

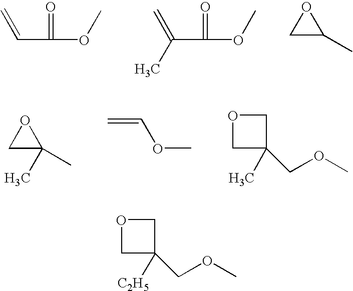

and still more preferable examples include:

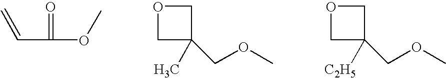

In the formula, each of L⁹ and L¹⁰ independently represents a single bond, —O—, —S—, —NR¹⁷— (R¹⁷ represents a hydrogen atom or methyl group), —CO₂—, —OCO₂— or —OCO—, more preferably a single bond, —O—, —CO₂—, —OCO₂— or —OCO—, and still more preferably a single bond, —O—, —CO₂— or —OCO—.

In the formula, S⁴ represents a divalent linking group selected from the group consisting of a single bond, —O—, —S—, —C(═O)—, —SO₂—, —NH—, —CH₂—, —CH═CH—, —C≡C— and combinations of them. S⁴ is preferably a divalent group containing 1 to 16 units of —CH₂— and 1 to 20 carbon atoms, more preferably containing 1 to 11 units of —CH₂— and 1 to 15 carbon atoms, and still more preferably containing 1 to 12 units of —CH₂— and 1 to 15 carbon atoms. Still more preferably, S⁴ is a divalent group selected from the group consisting of a single bond, —O—, —C(═O)—, —CH₂— and combinations of them, and having 1 to 12 carbon atoms. Hydrogen atom(s) of —NH—, —CH₂— and —CH═CH— may be replaced by substituent(s). Preferable examples of such substituent include halogen atoms, cyano group, nitro group, alkyl group having 1 to 6 carbon atoms, halogen-atom-substituted alkyl group having 1 to 6 carbon atoms, alkoxy group having 1 to 6 carbon atoms, acyl group having 2 to 6 carbon atoms, alkylthio group having 1 to 6 carbon atoms, acyloxy group having 2 to 6 carbon atoms, alkoxycarbonyl group having 2 to 6 carbon atoms, carbamoyl group, carbamoyl group with alkyl group having 2 to 6 carbon atoms and acylamino group having 2 to 6 carbon atoms, and more preferable examples include alkoxy group having 1 to 6 carbon atoms, and alkyl group having 1 to 6 carbon atoms.

Especially preferably, S⁴is a divalent group selected from the group consisting of combinations of —O— and CH₂—, containing 1 to 12 units of non-substituted CH₂—, and 0 to 4 units of —O—.

In the formula, each of n⁴ and n⁶ independently represents an integer of 0 to 5, more preferably 0 to 4, and still more preferably 0 to 3. The sum of n⁴ and n⁶ is preferably 1 to 10, more preferably 1 to 4, and still more preferably 1 to 3. Although n⁴ “R¹⁴”s and n⁶“R¹⁶”s may independently differ from each other, at least one of n⁴ “R¹⁴”s and n₆ “R¹⁶”s has a polymerizable group, Q.

In the formula, M represents the group shown below:

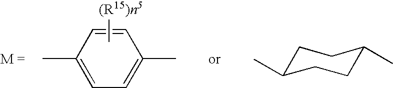

R¹⁵ represents —F, —Br, —Cl, —CH₃ or —OCH₃, preferably represents —F, —Cl, —CH₃ or —OCH₃, and still more preferably represents —F, —CH₃ or —OCH₃.

In the formula, n⁵represents an integer of 0 to 4, preferably 0 to 3, and still more preferably 0 to 2; and n⁵ “R¹⁵”s may differ from each other.

In the formula, each of L⁷ and L⁸ independently represents a single bond, —CO₂—, —OCO—, —CONR¹⁷— (R¹⁷ represents a hydrogen atom or methyl group), —NR¹⁸CO— (R¹⁸ represents a hydrogen atom or methyl group), —O—, —S—, —C(CH₃)₂—, —≡— or

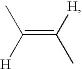

preferably a single bond, —CO₂—, —OCO—, —≡— or

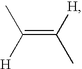

and still more preferably a single bond, —CO₂— or —OCO—.

In the formula, one of “R¹⁴”s and one of “R¹⁶”s are preferably substituted at the p-positions of L⁷ and L⁸, respectively.

For the embodiments where M represents:

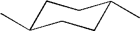

the linking groups on both ends of M preferably have a structure below:

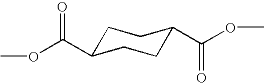

In formula (3), n represents an integer of 0 to 3, and preferably 0 to 2.

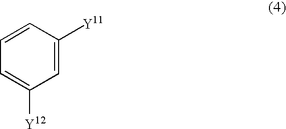

In formula (4), each of Y¹¹ and Y¹² independently represents formula (4-A), formula (4-B) or formula (4-C) below, and formula (4) contains at least one polymerizable group.

In formula (4), hydrogen atom(s) on the benzene ring may be replaced by substituent(s). Examples of such substituent include alkyl group, alkoxy group, aryloxy group, acyl group, alkoxycarbonyl group, acyloxy group, acylamino group, alkoxycarbonylamino group, alkylthio group, arylthio group, halogen atom and cyano. Among these substituents, alkyl group, alkoxy group, alkoxycarbonyl group, acyloxy group, halogen atom and cyano are more preferable, wherein alkyl group having 1 to 12 carbon atoms, alkoxy group having 1 to 12 carbon atoms, alkoxycarbonyl group having 2 to 12 carbon atoms, acyloxy group having 2 to 12 carbon atoms, halogen atom and cyano group are more preferable. The benzene ring may more preferably be non-substituted.

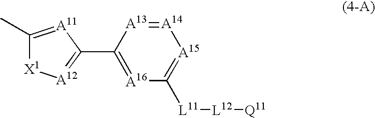

In formula (4-A), A¹¹, A¹², A¹³, A¹⁴, A¹⁵ and A¹⁶ each represent a methine or nitrogen atom; X¹ represents an oxygen atom, sulfur atom, methylene or imino; L¹¹ represents —O—, —O—CO—, —CO—O—, —O—CO—O—, —S—, —NH—, —SO₂—, —CH₂—, —CH═CH— or —C≡C—; L¹² represents a divalent linking group selected from the group consisting of —O—, —S—, —C(═O)—, —SO₂—, —NH—, —CH₂—, —CH═CH—, —C≡C— and combinations thereof wherein any of which having hydrogen atom(s) may have substituent(s) in place of the hydrogen atom(s); and Q¹¹ represents a polymerizable group or hydrogen atom.

Preferably, at least one of A¹¹ and A¹² is a nitrogen atom, and more preferably both of A¹¹ and A¹² are nitrogen atoms. Preferably, at least three of A¹³, A¹⁴, A¹⁵ and A¹⁶ are methines, and more preferably, all of them are methines. Non-substituted methine is preferable.

Examples of the substituent of methine represented by A¹³, A¹⁴, A¹⁵ or A¹⁶ include halogen atoms such as fluorine, chlorine, bromine and iodine atoms, cyano, nitro, alkyl group having 1 to 16 carbon atoms, alkenyl group having 2 to 16 carbon atoms, alkynyl group having 2 to 16 carbon atoms halogen-substituted alkyl group having 1 to 16 carbon atoms, alkoxy group having 1 to 16 carbon atoms, acyl group having 2 to 16 carbon atoms, alkylthio group having 1 to 16 carbon atoms, acyloxy group having 2 to 16 carbon atoms, alkoxycarbonyl group having 2 to 16 carbon atoms, carbamoyl group, carbamoyl with an alkyl group having 2 to 16 carbon atoms, and acylamino group having 2 to 16 carbon atoms. Halogen atoms, alkyl group having 1 to 6 carbon atoms, and halogen-substituted alkyl group having 1 to 6 carbon atoms are more preferable; halogen atoms, alkyl group having 1 to 4 carbon atoms, and halogen-substituted alkyl group having 1 to 4 carbon atoms are much more preferable; and halogen atoms, alkyl group having 1 to 3 carbon atoms, and trifluoromethyl are even much more preferable.

In formula (4-A), X¹ represents an oxygen atom, sulfur atom methylene to imino, and more preferably represents an oxygen atom.

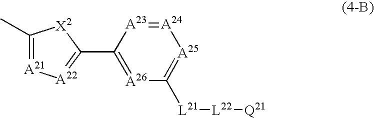

In formula (4-B), A²¹, A^{22,} A²³, A²⁴, A²⁵ and A²⁶ each independently represent a methine or nitrogen atom; X² represents an oxygen atom, sulfur atom, methylene or imino; L²¹ represents —O—, —O—CO—, —CO—O—, —O—CO—O—, —S—, —NH—, —SO₂—, —CH₂—, —CH═CH— or —C≡C—; L²² represents a divalent linking group selected from the group consisting of —O—, —S—, —C(═O)—, —SO₂—, —NH—, —CH₂—, —CH═CH—, —C≡C— and combinations thereof wherein any of which having hydrogen atom(s) may have substituent(s) in place of the hydrogen atom(s); and Q²¹ represents a polymerizable group or hydrogen atom.

Preferably, at least one of A²¹ and A²² is a nitrogen atom, and more preferably both of A²¹ and A²² are nitrogen atoms. Preferably, at least three of A²³, A²⁴, A²⁵ and A²⁶ are methines, and more preferably, all of them are methines. Non-substituted methine is preferable.

Examples of the substituent of methine represented by A²³, A²⁴, A²⁵ or A²⁶ include halogen atoms such as fluorine, chlorine, bromine and iodine atoms, cyano, nitro, alkyl group having 1 to 16 carbon atoms, alkenyl group having 2 to 16 carbon atoms, alkynyl group having 2 to 16 carbon atoms halogen-substituted alkyl group having 1 to 16 carbon atoms, alkoxy group having 1 to 16 carbon atoms, acyl group having 2 to 16 carbon atoms, alkylthio group having 1 to 16 carbon atoms, acyloxy group having 2 to 16 carbon atoms, alkoxycarbonyl group having 2 to 16 carbon atoms, carbamoyl group, carbamoyl with an alkyl group having 2 to 16 carbon atoms, and acylamino group having 2 to 16 carbon atoms. Halogen atoms, alkyl group having 1 to 6 carbon atoms, and halogen-substituted alkyl group having 1 to 6 carbon atoms are more preferable; halogen atoms, alkyl group having 1 to 4 carbon atoms, and halogen-substituted alkyl group having 1 to 4 carbon atoms are much more preferable; and halogen atoms, alkyl group having 1 to 3 carbon atoms, and trifluoromethyl are even much more preferable.

In formula (4-B), X² represents an oxygen atom, sulfur atom methylene to imino, and more preferably represents an oxygen atom.

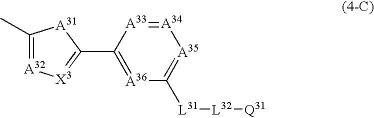

In formula (4-C), A³¹, A³², A³³, A³⁴, A³⁵ and A³⁶ each independently represents a methine or nitrogen atom; X³ represents an oxygen atom, sulfur atom, methylene or imino; L³¹ represents —O—, —O—CO—, —CO—O—, —O—CO—O—, —S—, —NH—, —SO₂—, —CH₂—, —CH═CH— or —C≡C—; L³² represents a divalent linking group selected from the group consisting of —O—, —S—, —C(═O)—, —SO₂—, —NH—, —CH₂—, —CH═CH—, —C≡C— and combinations thereof wherein any of which having hydrogen atom(s) may have substituent(s) in place of the hydrogen atom(s); and Q³¹ represents a polymerizable group or hydrogen atom.

Preferably, at least one of A³¹ and A³² is a nitrogen atom, and more preferably both of A³¹ and A³² are nitrogen atoms. Preferably, at least three of A³³, A³⁴, A³⁵ and A³⁶ are methines, and more preferably, all of them are methines.

The methine represented by A³³, A³⁴, A³⁵ or A³⁶ may have one or more substituents. Examples of the substituent include halogen atoms such as fluorine, chlorine, bromine and iodine atoms, cyano, nitro, alkyl group having 1 to 16 carbon atoms, alkenyl group having 2 to 16 carbon atoms, alkynyl group having 2 to 16 carbon atoms halogen-substituted alkyl group having 1 to 16 carbon atoms, alkoxy group having 1 to 16 carbon atoms, acyl group having 2 to 16 carbon atoms, alkylthio group having 1 to 16 carbon atoms, acyloxy group having 2 to 16 carbon atoms, alkoxycarbonyl group having 2 to 16 carbon atoms, carbamoyl group, carbamoyl with an alkyl group having 2 to 16 carbon atoms, and acylamino group having 2 to 16 carbon atoms. Halogen atoms, cyano, alkyl group having 1 to 6 carbon atoms, and halogen-substituted alkyl group having 1 to 6 carbon atoms are more preferable; halogen atoms, alkyl group having 1 to 4 carbon atoms, and halogen-substituted alkyl group having 1 to 4 carbon atoms are much more preferable; and halogen atoms, alkyl group having 1 to 3 carbon atoms, and trifluoromethyl are even much more preferable.

In formula (4-C), X³ represents an oxygen atom, sulfur atom methylene to imino, and more preferably represents an oxygen atom.

Preferably, L¹¹ in formula (4-A), L²¹ in formula (4-B) or L³¹ in formula (4-C) each represents —O—, —O—CO—, —CO—O—, —O—CO—O—, —CH₂—, —CH═CH— or —C≡C—, and more preferably represents —O—, —O—CO—, —CO—O—, —O—CO—O— or —CH₂—, wherein any of which containing hydrogen atom(s) may have substituent(s) in place of the hydrogen atom(s). Examples of such substituent include halogen atoms, cyano, nitro, alkyl group having 1 to 6 carbon atoms, halogen atom-substituted alkyl group having 1 to 6 carbon atoms, alkoxy group having 1 to 6 carbon atoms, acyl group having 2 to 6 carbon atoms, alkylthio group having 1 to 6 carbon atoms, acyloxy group having 2 to 6 carbon atoms, alkoxycarbonyl group having 2 to 6 carbon atoms, carbamoyl group, carbamoyl group with alkyl group having 2 to 6 carbon atoms, and acylamino group having 2 to 6 carbon atoms, wherein halogen atoms and alkyl group having 1 to 6 carbon atoms are more preferable.

Preferably, L¹² in formula (4-A), L²² in formula (4-B) or L³² in formula (4-C) is selected from the group consisting of —O—, —C(═O)—, —CH₂—, —CH═CH—, —C≡C— and combinations thereof. Preferably, L¹², L²² or L³² has 1 to 20 carbon atoms, more preferably 2 to 14 carbon atoms; preferably, L¹², L²² or L³² contains 1 to 14 units of —CH₂— and has 2 to 14 carbon atoms, and more preferably contains 2 to 12 units of —CH₂— and has 2 to 14 carbon atoms. Hydrogen atom(s) in —NH—, —CH₂ or —CH═CH— may be replaced with substituent(s). Examples of such substituent include halogen atoms, cyano, nitro, alkyl group having 1 to 6 carbon atoms, halogen atom-substituted alkyl group having 1 to 6 carbon atoms, alkoxy group having 1 to 6 carbon atoms, acyl group having 2 to 6 carbon atoms, alkylthio group having 1 to 6 carbon atoms, acyloxy group having 2 to 6 carbon atoms, alkoxycarbonyl group having 2 to 6 carbon atoms, carbamoyl group, carbamoyl group with alkyl group having 2 to 6 carbon atoms, and acylamino group having 2 to 6 carbon atoms, wherein alkoxy group having 1 to 6 carbon atoms and alkyl group having 1 to 6 carbon atoms are more preferable.

Q¹¹ in formula (4-A), Q²¹ in formula (4-B) or Q³¹ in formula (4-C) each represents a polymerizable group or hydrogen atom; and the definition of the polymerizable group is as same as that of Q in formula (3), as well as its preferable examples.

Examples of the polymer compound represented by formula (1) include, but are not limited to, those shown below.

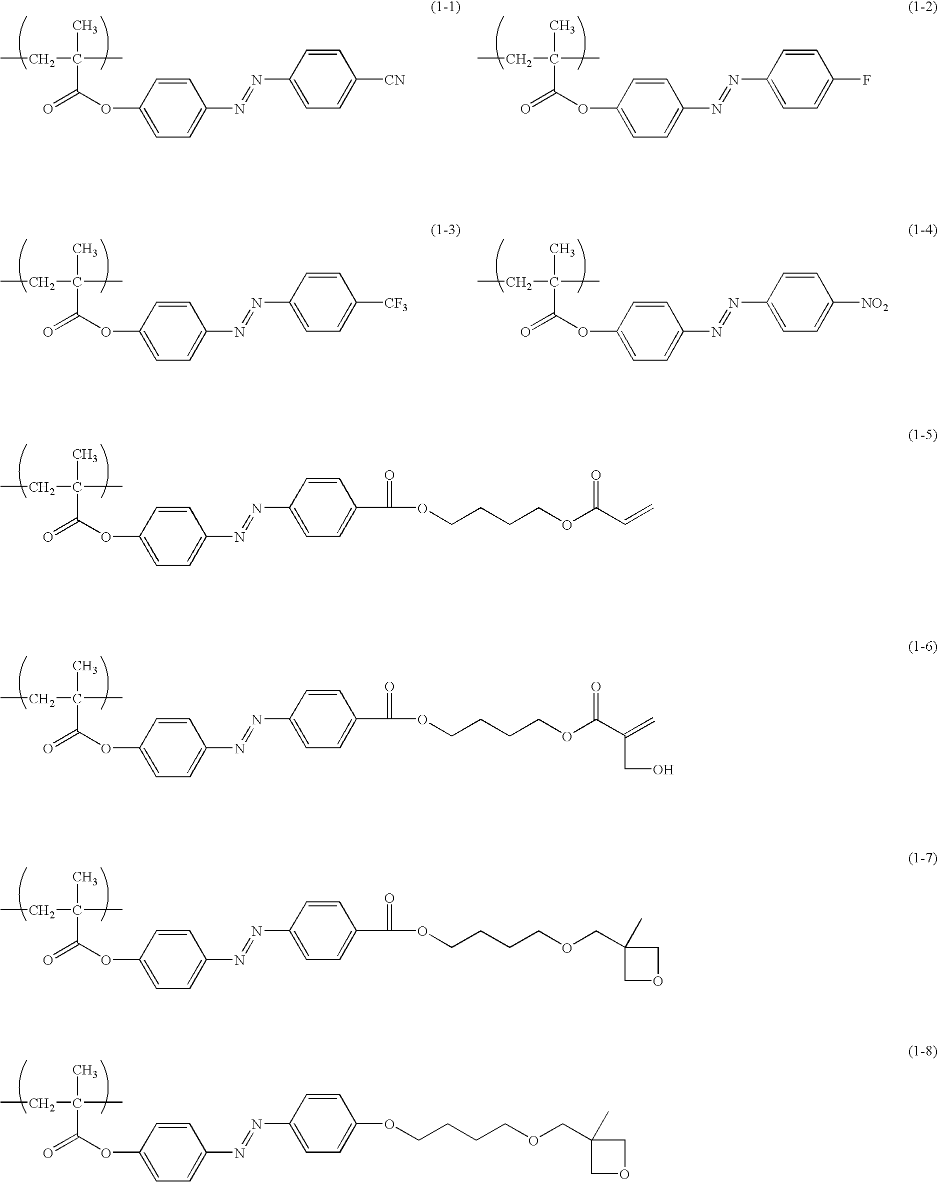

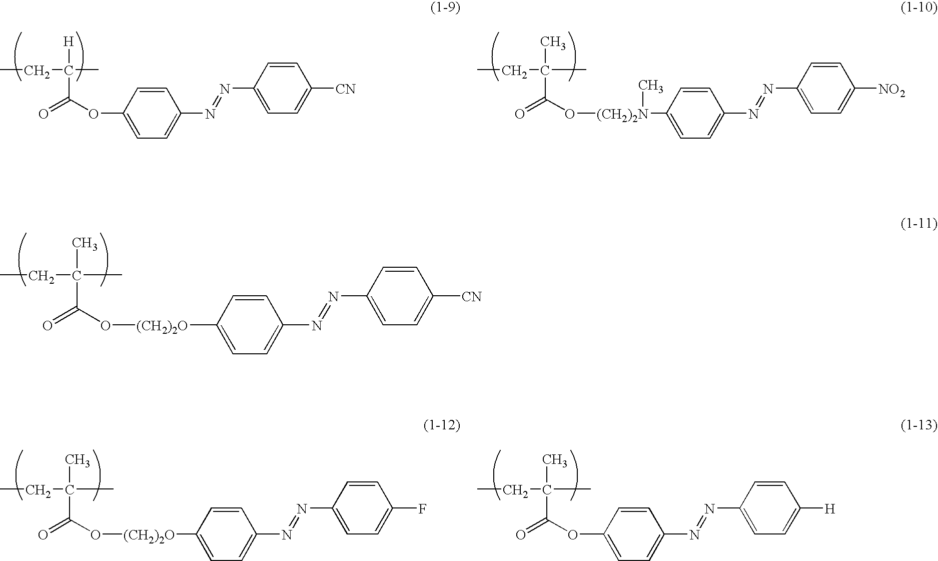

Examples of the compound represented by formula (2) include, but are not limited to, those shown below.

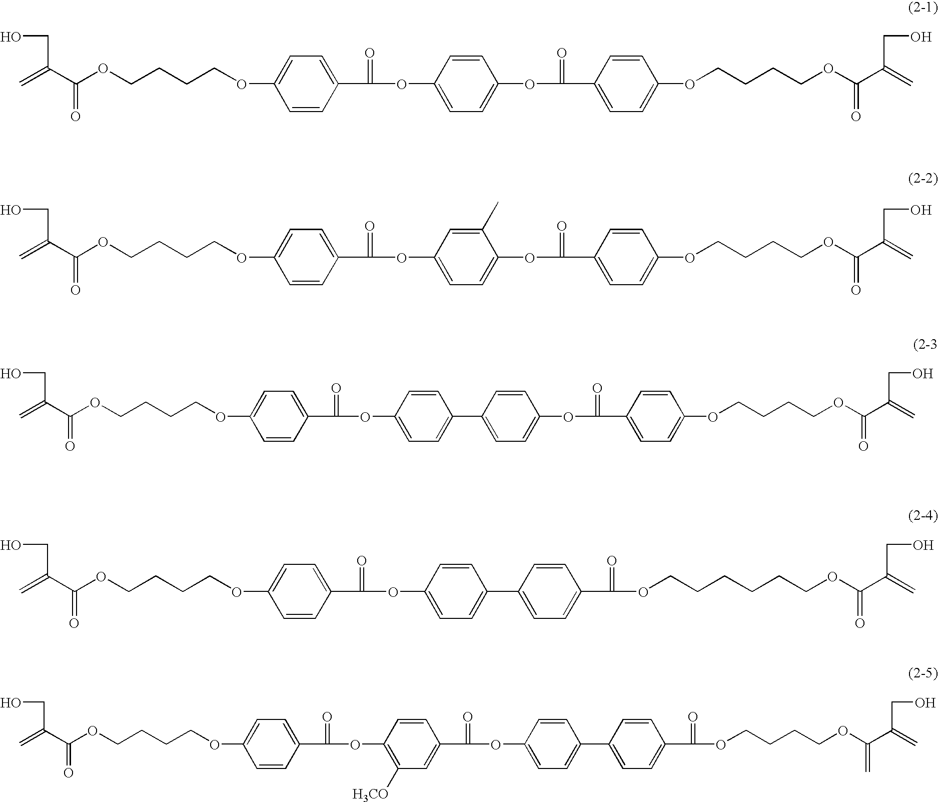

Examples of the compound represented by formula (3) include, but are not limited to, those shown below.

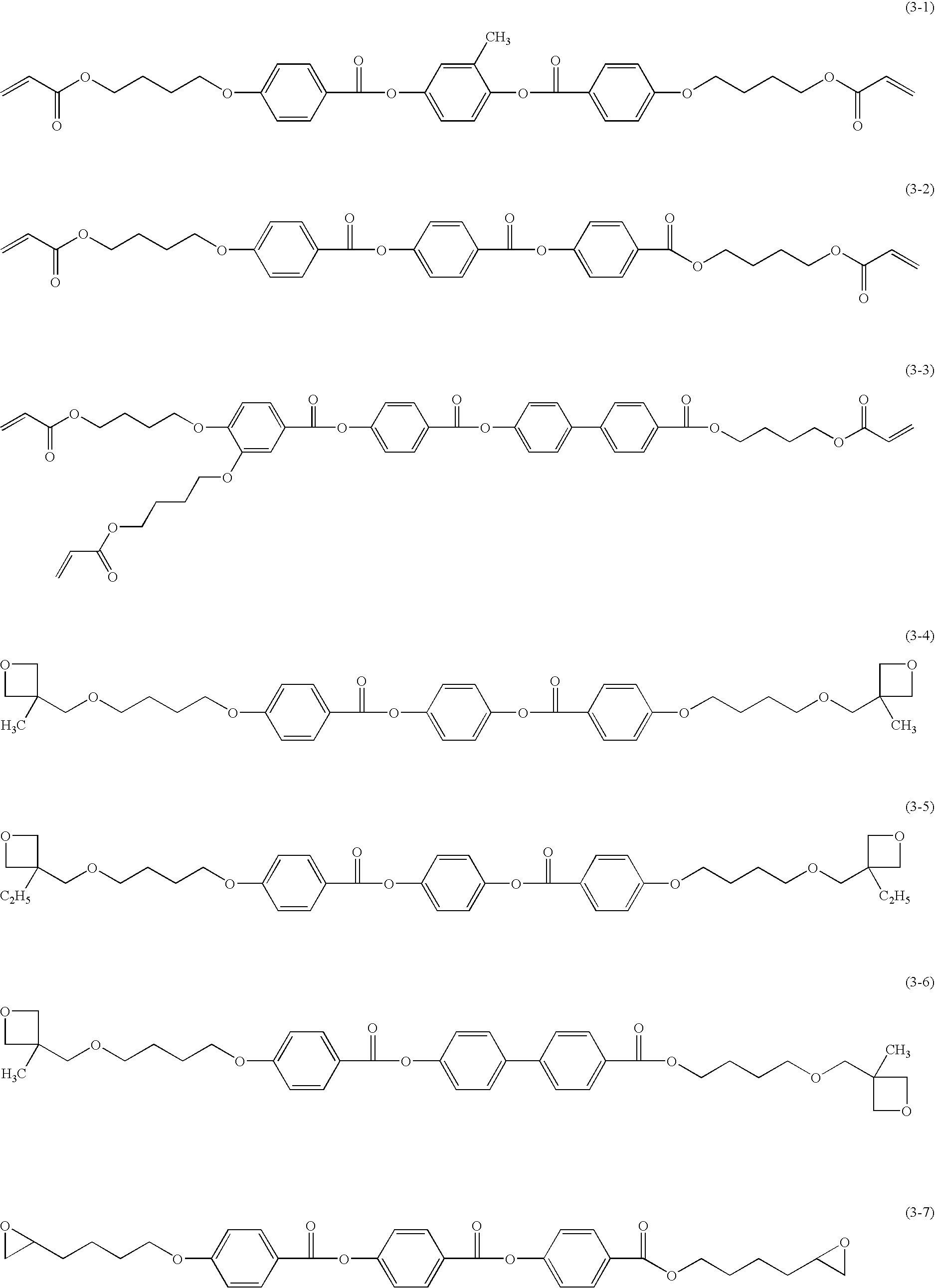

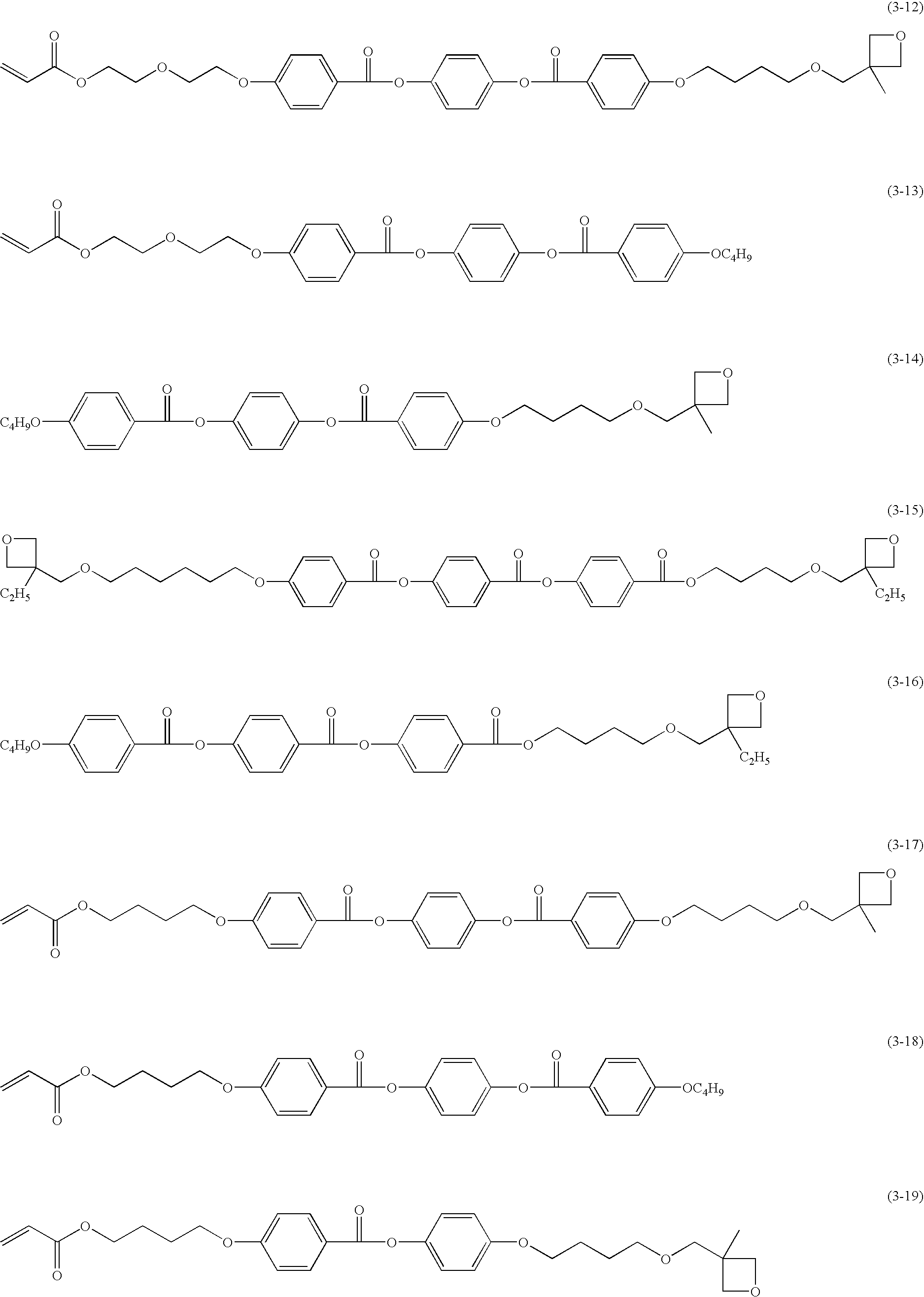

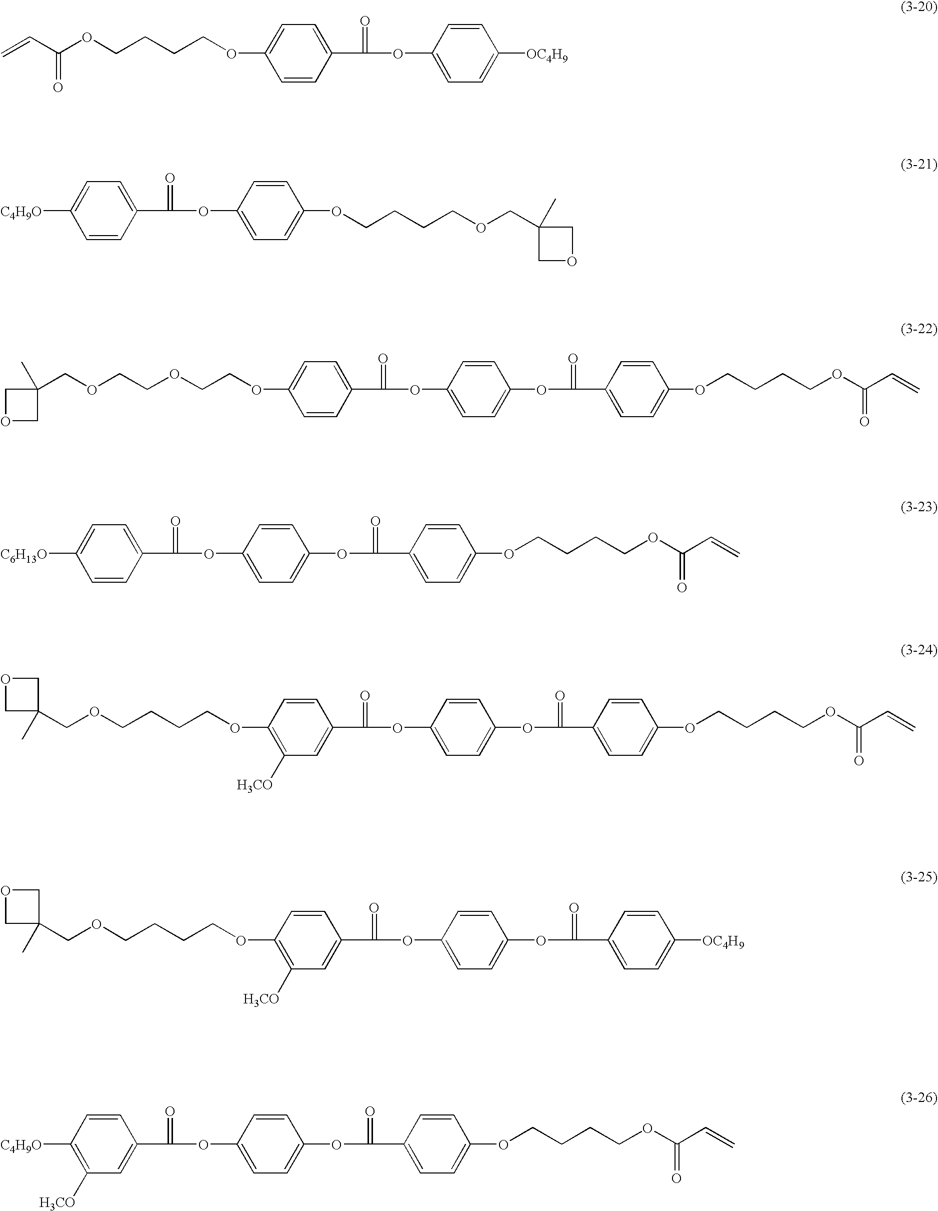

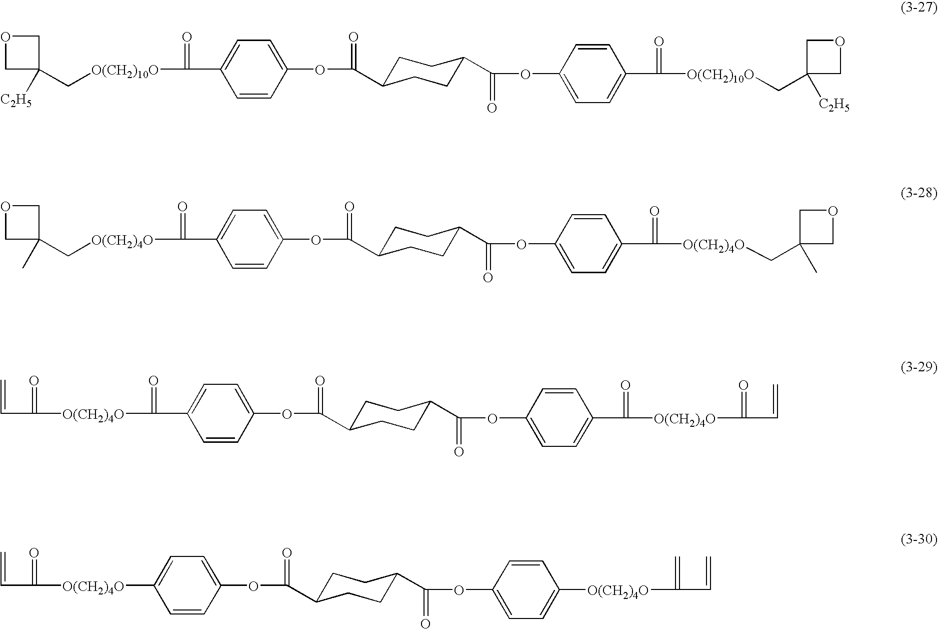

Examples of the compound represented by formula (4) include, but are not limited to, those shown below.

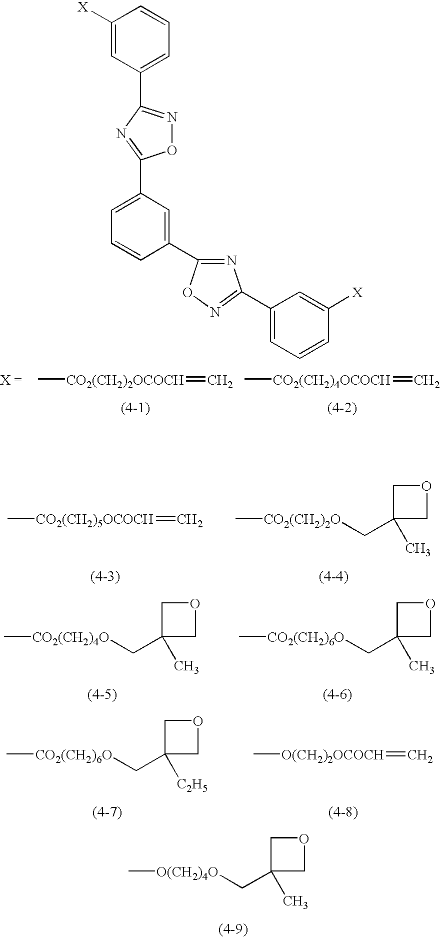

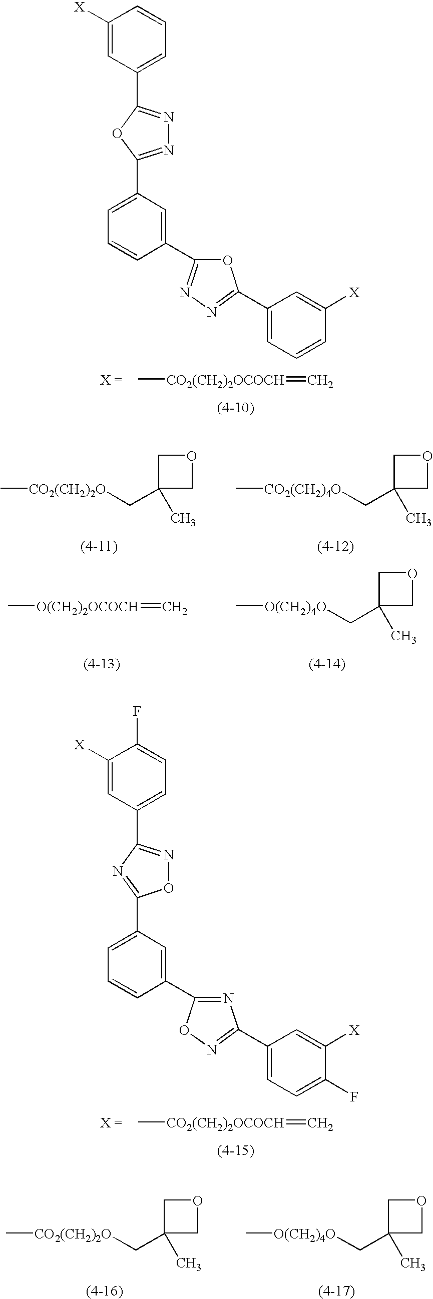

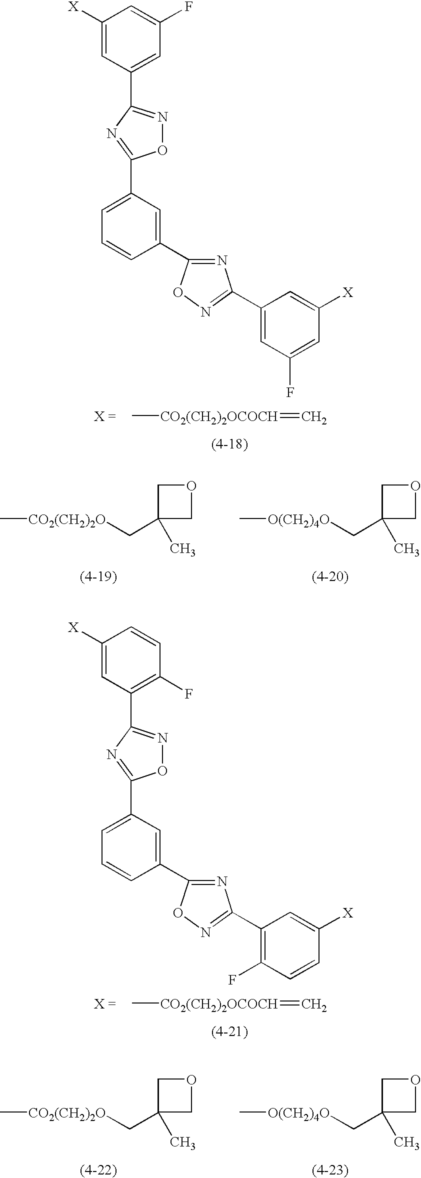

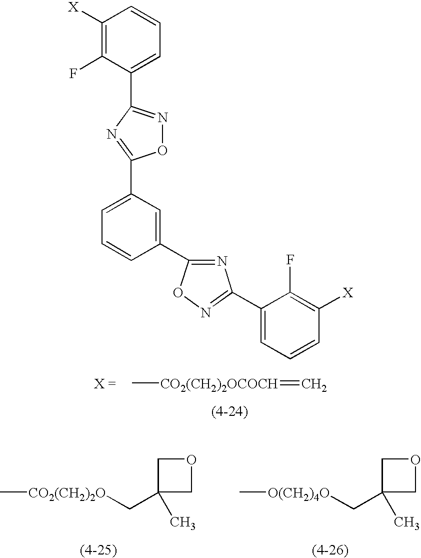

In the composition for photo-alignment film of the present invention, ratio of the amount of the polymer compound represented by formula (1) to the amount of the polymerizable compound (preferably selected from the compounds represented by formula (2), (3) or (4)) is preferably from 80 parts by mass/20 parts by mass to 5 parts by mass/95 parts by mass, and more preferably from 60 parts by mass/40 parts by mass to 5 parts by mass/95 parts by mass. The polymerizable compound (preferably selected from the compounds represented by formula (2), (3) or (4)) is preferably a liquid crystalline compound. One preferable example of the composition for photo-alignment film comprises the polymer compound represented by formula (1), at least one mono-functional monomer having only a single species of polymerizable group and at least one multi-functional monomer having two or more species of polymerizable group. In the example, the ratio of the amount of the mono-functional monomer to the amount of the multi-functional monomer is preferably from 10 parts by mass/90 parts by mass to 80 parts by mass/20 parts by mass, and more preferably from 20 parts by mass/80 parts by mass to 70 parts by mass/30 parts by mass.

Generally, the term “polymer” is used for compounds having a molecular weight equal to or more than 10000; and generally, compounds having a molecular weight of not less than 1000 and less than 10000 are called as a quasi-polymer. And compounds of which polymerization degree is from 2 to 200 are called as an oligomer, and they are distinguished from polymers (see “3-th additional edition Iwanami Dictionary of Physical and Chemical Science (IWANAMI RIKAGAKU JITEN)”, p. 449, edited by Bunishi Tamamushi et al., published by Iwanami Shoten in 1982. However, in the description, the term “polymer” indicates not only polymers but also quasi-polymers and examples of the polymer include any compounds having a molecular weight of equal to or more than 1000 and having a polymerization degree of equal to or more than 20.

### [Composition for Retardation Film]

The present invention relates also to a composition for retardation film comprising at least one polymer compound represented by formula (1), and at least one polymerizable compound represented by formula (2), (3) or (4).

Description on the formulas (1), (2), (3) and (4), and preferable ranges are similar to those described for the composition for photo-alignment film in the above.

### [Photo-Alignment Film]

The present invention relates also to a photo-alignment film formed of the composition for photo-alignment film of the invention. The composition is irradiated with light, and then the ability of controlling alignment of liquid crystal molecules develops in the composition. An exemplary method of producing a photo-alignment film of the present invention comprises applying the composition to a substrate to form a layer of the composition, and irradiating the layer of the composition with light. Then, the ability of controlling alignment develops in the layer, or in other words, the layer becomes an alignment film. According to this method, the composition for photo-alignment film is dissolved into a solvent, to thereby prepare a coating liquid. The solvent used herein is not specifically limited so far as they can dissolve the composition for photo-alignment film of the present invention, wherein solvents having relatively low vapor pressure at room temperature and high boiling point may be easy to handle for applying the coating liquid to a surface. Examples of the solvent include 1,1,2-trichloroethane, N-methyl pyrrolidone, butoxyethanol, γ-butyrolactone, ethylene glycol, polyethylene glycol monomethyl ether, propylene glycol, 2-pyrrolidone, N,N-dimethylformamide, phenoxyethanol, tetrahydrofuran, dimethylsulfoxide, methyl isobutyl ketone and cyclohexanone. Two or more organic solvents may be used in combination.

The coating liquid may be applied to a surface according to any known coating method such as a spin coating, die coating and gravure coating methods or any known printing methods such as flexographic printing and ink jet printing methods.

Next, the layer of the composition is irradiated with linear polarized light or irradiated with non-polarized light in an oblique direction; and then, the ability of controlling alignment develops in the layer. In this irradiation step, near ultraviolet of 350 nm to 450 nm is preferable. Examples of light source includes xenon lamp, high-pressure mercury lamp, ultrahigh-pressure mercury lamp and metal halide lamp. Ultraviolet radiation and visible light obtained from these light sources may be limited in range of wavelength to be irradiated, using an interference filter, color filter and so forth. Linear polarized light may be obtained by adopting a polarizing filter or polarizing prism to the light from these light sources. In this step, the irradiation energy may be from 10 mJ/cm² to 1000 mJ/cm², preferably from 20 mJ/cm² to 500 mJ/cm², and more preferably from 20 mJ/cm² to 300 mJ/cm². In this step, the light intensity is preferably from 10 to 1000 mW/cm², more preferably from 20 to 500 mW/cm², and much more preferably from 20 to 300 mW/cm².

The oblique direction herein means direction inclined by polar angle 0 (0<θ<90°) away from the normal line relative to the layer plane, wherein θ generally, and preferably, falls in the range from 20 to 80°, although not specifically limited.

Heating, subsequent to the irradiation of light, may progress the thermal polymerization, and is therefore preferable in terms of obtaining the photo-alignment film more durable against heat or the like. Temperature of heating is not specifically limited so far as it may be high enough to progress the polymerization, and is adjusted generally to the range from 50 to 240° C. or around, preferably to the range from 80 to 200° C. or around, and more preferably to the range from 80 to 190° C. or around. In the heat-assisted polymerization, the composition may be, or may not be, added with an initiator.

In terms of further improving the durability against light, heat and so forth, it is preferable to carry out irradiating the layer of the composition with non-polarized light after the step of irradiating the layer with linear polarized light or irradiating the layer with non-polarized light in an oblique direction, in place of, or before or after the heating, so as to progress polymerization and curing of the polymerizable compound in the layer. For the embodiments where the polymerization is progressed by irradiation of non-polarized light, the composition for photo-alignment film preferably contains a polymerization initiator preliminarily added thereto. Examples of the polymerization initiator include radical polymerization initiator and cationic polymerization initiator, each of which may be involved in thermal polymerization reaction making use of thermal polymerization initiator, and photo polymerization reaction making use of photo polymerization initiator. They may be selectable depending on the polymerizable group of the polymerizable ingredient in the composition.

Examples of the thermal polymerization initiator to be used in radical polymerizations include azobisisobutyronitrile. Examples of the photo-polymerization initiator to be used in radical polymerizations include α-carbonyl compounds (those described in U.S. Pat. Nos. 2,367,661 and 2,367,670), acyloin ethers (those described in U.S. Pat. No. 2,448,828), α-hydrocarbon-substituted aromatic acyloin compounds (those described in U.S. Pat. No. 2,722,512), polynuclear quinone compounds (those described in U.S. Pat. Nos. 3,046,127 and 2,951,758), combinations of triarylimidazole dimer and p-aminophenyl ketone (those described in U.S. Pat. No. 3,549,367), acrydine and phenazine compounds (those described in JPA No. S60-105667 and U.S. Pat. No. 4,239,850), and oxadiazole compounds (those described in U.S. Pat. No. 4,212,970).

Examples of the thermal polymerization initiator to be used in cationic polymerizations include benzylsulfonium salt compounds. Examples of the photo polymerization initiator include those of organic sulfonium base, iodonium salt base and phosphonium salt base. Examples of counter ion of these compounds include SbF₆⁻, PF₆⁻ and BF₆⁻.

Amount of addition of the polymerization initiator is preferably 0.1 to 10% by mass in the composition, more preferably 0.1 to 8% by mass, and still more preferably 0.1 to 7% by mass.

In this irradiation step to progress the polymerization, ultraviolet light is preferable. In this step, the irradiation energy is preferably from 10 mJ/cm² to 10 J/cm², and more preferably from 50 mJ/cm² to 5 J/cm². In this step, the light intensity is preferably from 10 to 1000 mW/cm², more preferably from 20 to 500 m W/cm², and much more preferably from 20 to 350 mW/cm². Light to be used in this step preferably has a peak within the range from 250 to 450 nm, and more preferably within the range from 300 to 410 nm. To promote the polymerization, irradiation with light may be carried out under a nitrogen atmosphere or heat.

The thickness of the photo-alignment film is preferably from 10 to 500 nm around, more preferably from 10 to 300 nm around, and even more preferably from 10 to 100 nm around.

### [Retardation Film]

The present invention relates still also to a retardation film formed of the composition for retardation film. Being irradiated with polarized light, the composition aligns; and then birefringence develops in the composition. A method of producing the retardation film of the present invention is similar to that for the photo-alignment film described in the above, wherein it is similarly preferable to provide a heating step successive to the irradiation of polarized light or non-polarized light in an oblique direction, and to carry out the step of irradiation of non-polarized light in place of, or before or after the heating, in terms of obtaining durability.

The retardation film of the present invention, specifiable in the direction of alignment thereof by irradiation of polarized light or non-polarized light in an oblique direction, may be formed on a substrate even in the absence of alignment film, so that fine patterned retardation film may be produced without using a technique such as patterning.

Thickness of the retardation film to be formed may vary depending on applications, wherein the thickness may generally be adjusted preferably to the range from 0.1 to 20 μm, and more preferable to the range from 0.2 to 15 μm.

### [Liquid Crystal Cell and Liquid Crystal Display Device]

The present invention relates to a liquid crystal cell having a liquid crystal composition held between a pair of substrates, and having the photo-alignment film of the present invention on the inner surface of at least one of the pair of substrates; and a liquid crystal display device comprising the liquid crystal cell. The photo-alignment film of the present invention is useful as a homogeneous alignment film capable of aligning liquid crystal molecules horizontally, and is therefore suitable for embodiments of IPS-mode liquid crystal display device.

The present invention relates also to a liquid crystal display device comprising the retardation film of the present invention. One example of the liquid crystal display device of the present invention comprises a pair of substrates, and a liquid crystal layer held therebetween, and the retardation film formed of the composition of the present invention disposed on the inner surface of at least one of the pair of substrates. Of course, embodiments having the retardation film disposed outside the substrates are included in the scope of the present invention.

Either of organic materials and inorganic materials may be adoptable as materials composing the substrates. Specific examples include inorganic materials such as glass, silicon and so forth; and organic materials such as polyethylene terephthalate, polycarbonate, triacetyl cellulose and so forth. These substrates may have electrode layers such as those composed of ITO, Cr, Al and so forth provided thereto, and may have also color filter layers formed thereon.

### EXAMPLES

The invention is described more concretely with reference to the following Examples, in which the material and the reagent used, their amount and the ratio, the details of the treatment and the treatment process may be suitably modified or changed not overstepping the sprit and the scope of the invention. Accordingly, the invention should not be limited by the Examples mentioned below.

### Examples 1 to 9 and Comparative Example 1

### Production of Photo-Alignment Film and Evaluation of Orientation of Liquid Crystal

1,1,2-Trichloroethane solutions having a formulation respectively shown in Table 1 were prepared, each sample solution was applied to a surface of a glass substrate according to a spin coating method (3500 rpm, 20 seconds), and the layer of the solution was irradiated with polarized ultraviolet light of 365 nm at an energy of 100 mJ/cm² from in the direction along the normal line relative to the layer plane. According to Example 1, after carrying out irradiation with polarized light, the layer was heated on a hot plate at 140° C. for 5 minutes, and irradiated with non-polarized light in the atmosphere at 70° C., using a high-pressure mercury lamp having an energy of 140 mJ/cm² for 10 seconds, to thereby fabricate a photo-alignment film.

According to Examples 2 to 9 and Comparative Example 1, after carrying out irradiation with polarized light, the layer was heated on a hot plate at 100° C. under a nitrogen atmosphere for 10 minutes, to thereby fabricate photo-alignment films.

An isopropyl alcohol solution having a formulation shown in Table 2 was prepared. The solution was applied to a surface of each of the photo-alignment films fabricated in Examples 1 to 9 and Comparative Example 1 according to a spin coating method (2000 rpm, 20 seconds), heated at 80° C. for 10 seconds, cooled to room temperature, and orientation of the liquid crystal was observed. Visual observation revealed that all of samples prepared in Examples 1 to 5, 7 to 9 and Comparative Example 1 showed desirable orientation of the liquid crystal, but Example 6 was found to be slightly poor in orientation. The Exemplary Compound (1-1) used herein is the compound described in Japanese Patent No. 2990270.

TABLE 1

| | |
|---|---|
| Example 1 | Compound (1-1): 0.85 parts by mass, |
| | Compound (3-15): 0.69 parts by mass, |
| | Compound (3-21): 0.38 parts by mass, |
| | 50% propylene carbonate solution of triallylsulfonium |
| | hexafluorophosphate as a polymerization initiator (from |
| | Aldrich): 0.08 parts by mass, and |
| | 1,1,2-trichloroethane: 98 parts by mass |
| Example 2 | Compound (1-1): 1.14 parts by mass, |
| | Compound (3-1): 0.762 parts by mass, |
| | 2,2′-azobis(2,4-dimethyl valeronitrile) as a polymerization |
| | initiator: 0.0952 parts by mass, and 1,1,2-trichloroethane: |
| | 98 parts by mass |
| Example 3 | Compound (1-1): 1.14 parts by mass, |
| | Compound (3-2): 0.762 parts by mass, |
| | 2,2′-azobis(2,4-dimethyl valeronitrile) as a polymerization |
| | initiator: 0.0952 parts by mass, and 1,1,2-trichloroethane: |
| | 98 parts by mass |
| Example 4 | Compound (1-1): 1.14 parts by mass, |
| | Compound (3-3): 0.762 parts by mass, |
| | 2,2′-azobis(2,4-dimethyl valeronitrile) as a polymerization |
| | initiator: 0.0952 parts by mass, and 1,1,2-trichloroethane: |
| | 98 parts by mass |
| Example 5 | Compound (1-1): 1.14 parts by mass, |
| | Compound (4-1): 0.762 parts by mass, |
| | 2,2′-azobis(2,4-dimethyl valeronitrile) as a polymerization |
| | initiator: 0.0952 parts by mass, and 1,1,2-trichloroethane: |
| | 98 parts by mass |
| Example 6 | Compound (1-1): 1.14 parts by mass, |
| | dipentaerythritol hexaacrylate: 0.762 parts by mass, |
| | 2,2′-azobis(2,4-dimethyl valeronitrile) as a polymerization |
| | initiator: 0.0952 parts by mass, and 1,1,2-trichloroethane: |
| | 98 parts by mass |
| Example 7 | Compound (1-4): 1.14 parts by mass, |
| | Compound (3-1): 0.762 parts by mass, |
| | 2,2′-azobis(2,4-dimethyl valeronitrile) as a polymerization |
| | initiator: 0.0952 parts by mass, and 1,1,2-trichloroethane: |
| | 98 parts by mass |
| Example 8 | Compound (1-4): 1.14 parts by mass, |
| | Compound (3-2): 0.762 parts by mass, |
| | 2,2′-azobis(2,4-dimethyl valeronitrile) as a polymerization |
| | initiator: 0.0952 parts by mass, and 1,1,2-trichloroethane: |
| | 98 parts by mass |
| Example 9 | Compound (1-4): 1.14 parts by mass, |
| | Compound (3-3): 0.762 parts by mass, |
| | 2,2′-azobis(2,4-dimethyl valeronitrile) as a polymerization |
| | initiator: 0.0952 parts by mass, and 1,1,2-trichloroethane: |
| | 98 parts by mass |
| Comparative | Compound (1-1): 2 parts by mass, and 1,1,2- |
| Example 1 | trichloroethane: 98 parts by mass |

TABLE 2
MLC-16000-l00 (liquid crystal compound from MERCK): 34.9 parts by mass, air interface
controlling agent (A): 0.0349 parts by mass, and isopropyl alcohol: 65 parts by mass

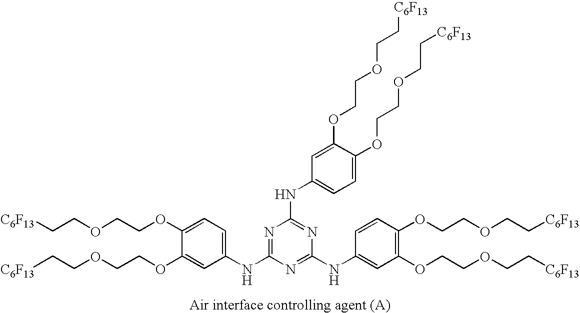

### Examples 10 to 18 and Comparative Example 2

### Preparation of Photo-Alignment Film and Evaluation of Orientation of Liquid Crystal

Each of the photo-alignment films fabricated in Example 1 to 9 and Comparative Example 1 was irradiated with non-polarized ultraviolet radiation at an energy of 30 J/cm² in the direction along the normal line relative to the film plane, to thereby fabricate photo-alignment films of Examples 10 to 18 and Comparative Example 2, respectively.

Next, an isopropyl alcohol solution of the liquid crystal composition shown in Table 2 was applied to a surface of each of the photo-alignment films fabricated in Examples 10 to 18 and Comparative Example 2 according to a spin coating method (2000 rpm, 20 seconds), heated at 80° C. for 10 seconds, cooled to room temperature, and orientation of the liquid crystal was observed. Comparison of orientation of liquid crystal in Examples 10 to 18 and Comparative Example 2, as compared with that in Examples 1 to 6 and Comparative Example 1, revealed that the photo-alignment films of Examples 10 to 18 were improved in light resistance as compared with the conventional photo-alignment film shown in Comparative Example 2.

### Example 19

A method of synthesizing Compound (2-2), as an example of liquid crystal compound represented by formula (2) is shown below.

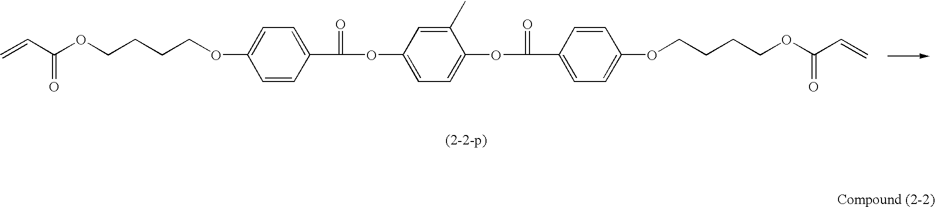

An intermediate (2-2-p) of the Exemplary Compound (2-2) was synthesized similarly to the compound described in WO93/22397.

Under cooling on ice, 1,4-diazabicyclo[2.2.2]octane (1.95 mmol) was added to 1.2 ml of an aqueous solution of paraformaldehyde (3.25 mmol), the mixture was stirred for 15 minutes, 4.5 ml of dimethylacetamide solution of (2-2-p) (0.811 mmol) was added, and the mixture was stirred at 40° C. for 7 hours. After completion of the reaction, the mixture was added with ethyl acetate, the organic layer was washed twice with dilute hydrochloric acid, and further washed once with water. The organic layer was dried over magnesium anhydride, the solvent was vaporized off under reduced pressure, the residue was purified by column chromatography (eluant: hexane/ethyl acetate=2/3 to l/2), to thereby obtain Compound (2-2) (yield: 27.3%).

¹HNMR(300 MHz,CDCl₃): δ 1.8-2.0(m,8H), 2.2-2.3(s,3H), 4.0-4.4(m,12H), 5.8-5.9(s,2H), 6.2-6.3(s,2H), 6.8-7.2(m,7H), 8.1-8.2 (m, 4H).

Compound (2-2) was found to cause phase transition to isotropic phase at 109° C., and was found to cause phase transition to nematic phase when the temperature is lowered to 105° C.

The coating liquid was prepared similarly to Example 2 except that Compound (2-2) was used in place of Compound (3-1), the alignment film was similarly fabricated, and the light was irradiated in the same manner as Example 10 and so forth. The photo-alignment film showed desirable light resistance similarly to as in the above-described Examples.

### Examples 20 to 26 and Comparative Example 3

### Fabrication of Retardation Film

Tetrahydrofuran solutions having a formulation respectively shown in Table 3 were prepared, each sample solution was applied to a surface of a glass substrate according to a spin coating method (2000 rpm, 20 seconds), and the layer of the solution was irradiated with polarized ultraviolet light of 365 nm at an energy of 100 mJ/cm² from in the direction along the normal line relative to the layer plane. Next, the layer was heated on a hot plate at 180° C. for 5 minutes, and then irradiated with non-polarized light in the atmosphere at 70° C. using a high-pressure mercury lamp having an energy of 140 mJ/cm² for 10 seconds, to thereby fabricate retardation films of Examples 20 to 26.

On the other hand, a coating liquid was prepared similarly to as described in Example 2 of JPA No. 2006-308878, the coating liquid was applied to a surface of a glass substrate according to a spin coating (1000 rpm, 30 seconds), and the layer of the solution was irradiated with non-polarized ultraviolet light of 405 nm at an energy of 1530 mJ/cm² in the direction along the normal line relative to the layer plane. The film was then heated on a hot plate at 190° C. for 10 minutes, to thereby fabricate a retardation film of Comparative Example 3.

TABLE 3

| | Composition |
|---|---|
| Example | Compound (1-1): 11.4 parts by mass, |
| 20 | Compound (3-1): 7.62 parts by mass, |
| | 2,2′-Azobis(2,4-dimethyl valeronitrile) as a polymerization initiator: 0.952 parts by mass, and |
| | tetrahydrofuran: 80.03 parts by mass |
| Example | Compound (1-1): 11.4 parts by mass, |
| 21 | Compound (3-3): 7.62 parts by mass, |
| | 2,2′-Azobis(2,4-dimethyl valeronitrile) as a polymerization initiator: 0.952 parts by mass, and |
| | tetrahydrofuran: 80.03 parts by mass |
| Example | Compound (1-1): 11.5 parts by mass, |
| 22 | Compound (3-4): 7.67 parts by mass, |
| | 50% propylene carbonate solution of triallylsulfonium hexafluorophosphate (from Aldrich) as a |
| | polymerization initiator: 0.8 parts by mass, and |
| | tetrahydrofuran: 80.03 parts by mass |
| Example | Compound (1-1): 11.5 parts by mass, |
| 23 | Compound (3-15): 7.67 parts by mass, |
| | 50% propylene carbonate solution of triallylsulfonium hexafluorophosphate (from Aldrich) as a |
| | polymerization initiator: 0.8 parts by mass, and |
| | tetrahydrofuran: 80.03 parts by mass |
| Example | Compound (1-1): 11.4 parts by mass, |
| 24 | Compound (4-2): 7.62 parts by mass, |
| | 2,2′-Azobis(2,4-dimethyl valeronitrile) as a polymerization initiator: 0.952 parts by mass, and |
| | tetrahydrofuran: 80.03 parts by mass |
| Example | Compound (1-3): 11.4 parts by mass, |
| 25 | Compound (3-1): 7.62 parts by mass, |
| | 2,2′-Azobis(2,4-dimethyl valeronitrile) as a polymerization initiator: 0.952 parts by mass, and |
| | tetrahydrofuran: 80.03 parts by mass |
| Example | Compound (1-3): 11.4 parts by mass, |
| 26 | Compound (3-3): 7.62 parts by mass, |
| | 2,2′-Azobis(2,4-dimethyl valeronitrile) as a polymerization initiator: 0.952 parts by mass, and |
| | tetrahydrofuran: 80.03 parts by mass |
| Comparative | Comparative Compound (PA): 19.5 parts by mass, |
| Example 3 | Comparative Compound (A): 0.5 parts by mass, and |
| *1 | cyclohexanone: 80 parts by mass |

*1: The method described in JPA No. 2006-308878.

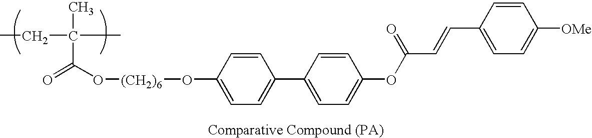

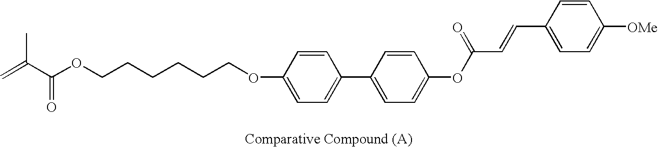

### Examples 20 to 26 and Comparative Example 3

### Evaluation of Retardation Film

The obtained retardation films were subjected to measurement of in-plane retardation Re at 550 nm, using an automatic birefringence meter (KOBRA-21ADH, from Oji Scientific Instruments).

The obtained glass substrates having the retardation layer formed thereon were then irradiated by non-polarized ultraviolet radiation at an energy of 30 J/cm² in the direction along the normal line relative to the layer plane, and were again subjected to measurement of retardation in plane Re at 550 nm.

TABLE 4

| | Re [nm] as fabricated | | Re [nm] after | |
|---|---|---|---|---|
| | (before irradiation with | | irradiated with | |
| | non-polarized light) | | non-polarized light at | |
| | (at 550 nm) | | 30 J/cm² (at 550 nm) | |
| Example 20 | 161 | nm | 160 | nm |
| Example 21 | 105 | nm | 105 | nm |
| Example 22 | 88 | nm | 87 | nm |
| Example 23 | 93 | nm | 91 | nm |
| Example 24 | 99 | nm | 98 | nm |
| Example 25 | 125 | nm | 123 | nm |
| Example 26 | 108 | nm | 107 | nm |
| Comparative | 112 | nm | 82 | nm |
| Example 3 | | | | |

It can be understood from the results shown in Table, that the retardation films of the present invention are excellent in stability against light (show excellent light resistance).

What is claimed is:

1. A composition for photo-alignment film comprising at least one polymer compound represented by formula (1) below, and at least one polymerizable compound: 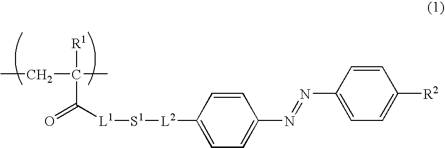
where, R¹ represents a hydrogen atom or methyl group; L¹ represents —O—, —NR³— (R³ represents a hydrogen atom or methyl group) or —S—; S¹ represents a divalent linking group selected from the group consisting of a single bond, —O—, —S—, —C(═O)—, —SO₂—, —NH—, —CH₂—, —CH═CH—, —C≡C— and combinations thereof, wherein any of which having hydrogen atom(s) may have substituent(s) in place of the hydrogen atom(s); L represents a single bond, —O—, —NR⁴— (R⁴ represents a hydrogen atom or methyl group), —S—, —OCO₂—, —CO₂— or —OCO—; R² represents a hydrogen atom, non-substituted or substituted alkylene group, —CN, —NO₂, non-substituted or substituted alkoxy group, —F, —Br, —Cl, —CF₃, —CO₂R⁵ (R⁵ represents a non-substituted or substituted alkyl group), —≡—R⁶ (R⁶ represents a hydrogen atom or non-substituted or substituted alkyl group) or 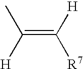
where R⁷ represents a hydrogen atom, or substituted or non-substituted alkyl group.

2. The composition for photo-alignment film of claim 1, wherein said polymerizable compound is a compound having two or more polymerizable groups.

3. The composition for photo-alignment film of claim 1, comprising two or more polymerizable compounds, at least one of which being a compound having one polymerizable group, and at least other one of which being a compound having two or more polymerizable groups.

4. The composition for photo-alignment film of claim 1, wherein said at least one polymerizable compound is a compound represented by formula (2), (3) or (4): 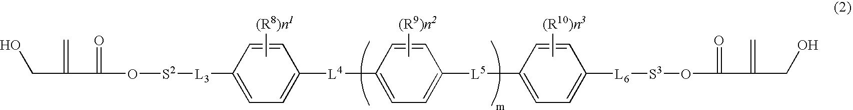
where each of R⁸, R⁹and R¹⁰ independently represents —F, —Br, —Cl, —CH₃ or —OCH₃ and is same or different at each occurrence; each of n¹ to n³ independently represents an integer of 0 to 4; each of S² and S³ independently represents a divalent linking group selected from the group consisting of a single bond, —O—, —S—, —C(═O)—, —SO₂—, —NH—, —CH₂—, —CH═CH— and —C≡C—, and combinations of them, wherein any of which containing hydrogen atom(s) may have substituent(s) in place of the hydrogen atom(s); each of L³ and L⁶ independently represents a single bond, —O—, —S—, —NR¹¹— (R¹¹ represents a hydrogen atom of methyl group), —CO₂—, —OCO₂— or —OCO—; each of L⁴ and L⁵ independently represents a single bond, —CO₂—, —OCO—, —CONR¹² (R¹² represents a hydrogen atom or methyl group), —NR¹³CO— (R¹³ represents a hydrogen atom or methyl group), —O—, —S—, —C(CH₃)₂—, —≡— or 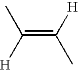
where m represents an integer of 0 to 3; 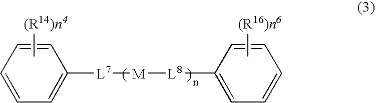
where each of R¹⁴ and R¹⁶ independently represents Q-L⁹-S⁴-L¹⁰-, where Q represents a hydrogen atom or polymerizable group, each of L⁹ and L¹⁰ independently represents a single bond, —O—, —S—, —NR¹⁷— (R¹⁷ represents a hydrogen atom or methyl group), —CO₂—, —OCO₂— or —OCO—, and S⁴ represents a divalent linking group selected from the group consisting of a single bond, —O—, —S—, —C(═O)—, —SO₂—, —NH—, —CH₂—, —CH═CH—, —C≡C— and combinations of them, wherein any of which having hydrogen atom(s) may have substituent(s) in place of the hydrogen atom(s); each of n⁴ and n⁶ independently represents an integer of 0 to 5 and the sum of n⁴ and n⁶ is from 1 to 10, n⁴ “R¹⁴”s and n⁶ “R¹⁶”s may independently differ from each other, at least one of n⁴ “R¹⁴”s and n⁶ “R¹⁶”s has a polymerizable group Q; M represents the group shown below: 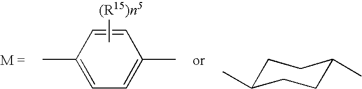
where R¹⁵ represents —F, —Br, —Cl, —CH₃ or —OCH₃, n⁵ represents an integer of 0 to 4, and n⁵ “R¹⁵”s may differ from each other; each of L⁷ and L⁸ independently represents a single bond, —CO₂—, —OCO—, —CONR¹⁷— (R¹⁷ represents a hydrogen atom or methyl group), —NR¹⁸CO— (R¹⁸ represents a hydrogen atom or methyl group), —O—, —S—, —C(CH₃)₂—, —≡— or 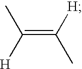
and n represents an integer from 0 to 3; provided that formula (3) has at least one polymerizable group; 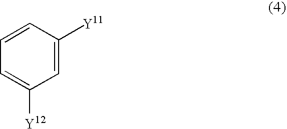
where each of Y¹¹ and Y¹² independently represents formula (4-A), formula (4-B) or formula (4-C) below, provided that formula (4) has at least one polymerizable group: 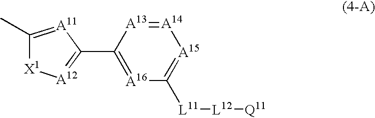
where A¹¹, A¹², A¹³, A¹⁴, A¹⁵ and A¹⁶ each represent a methine or nitrogen atom; X¹ represents an oxygen atom, sulfur atom, methylene or imino; L¹¹ represents —O—, —O—CO—, —CO—O—, —O—CO—O—, —S—, —NH—, —SO₂—, —CH₂—, —CH═CH— or —C≡C—; L¹² represents a divalent linking group selected from the group consisting of —O—, —S—, —C(═O)—, —SO₂—, —NH—, —CH₂—, —CH═CH—, —C≡C— and combinations thereof wherein any of which having hydrogen atom(s) may have substituent (s) in place of the hydrogen atom(s); and Q¹¹ represents a polymerizable group or hydrogen atom; 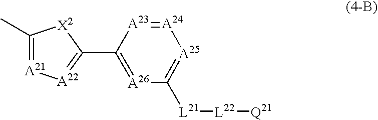
where A²¹, A²², A²³, A²⁴, A²⁵ and A²⁶ each independently represent a methine or nitrogen atom; X² represents an oxygen atom, sulfur atom, methylene or imino; L²¹ represents —O—, —O—CO—, —CO—O—, —O—CO—O—, —S—, —NH—, —SO²—, —CH₂—, —CH═CH— or —C≡C—; L²² represents a divalent linking group selected from the group consisting of —O—, —S—, —C(═O)—, —SO₂—, —NH—, —CH₂—, —CH═CH—, —C≡C— and combinations thereof wherein any of which having hydrogen atom(s) may have substituent(s) in place of the hydrogen atom(s); and Q²¹ represents a polymerizable group or hydrogen atom; 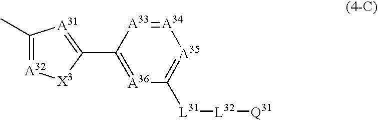
where A³¹, A³², A³³, A³⁴, A³⁵ and A³⁶ each independently represents a methine or nitrogen atom; X³ represents an oxygen atom, sulfur atom, methylene or imino; L³¹ represents —O—, —O—CO—, —CO—O—, —O—CO—O—, —S—, —NH—, —SO₂—, —CH₂—, —CH═CH— or —C≡C—; L₃₂ represents a divalent linking group selected from the group consisting of —O—, —S—, —C(═O)—, —SO₂—, —NH—, —CH₂—, —CH═CH—, —C≡C— and combinations thereof wherein any of which having hydrogen atom(s) may have substituent(s) in place of the hydrogen atom(s); and Q³¹ represents a polymerizable group or hydrogen atom.

5. The composition for photo-alignment film of claim 1, further comprising a polymerization initiator.

6. A composition for retardation film comprising at least one polymer compound represented by formula (1) below: 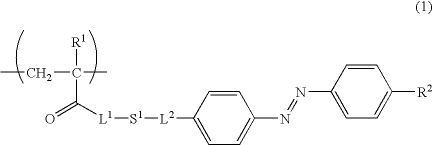
where, R¹ represents a hydrogen atom or methyl group; L¹ represents —O—, —NR³— (R³ represents a hydrogen atom or methyl group) or —S—; S¹ represents a divalent linking group selected from the group consisting of a single bond, —O—, —S—, —C(═O)—, —SO₂—, —NH—, —CH₂—, —CH═CH—, —C≡C— and combinations thereof, wherein any of which having hydrogen atom(s) may have substituent(s) in place of the hydrogen atom(s); L² represents a single bond, —O—, —NR⁴— (R⁴ represents a hydrogen atom or methyl group) , —S—, —OCO₂—, —CO₂— or —OCO—; R² represents a hydrogen atom, non-substituted or substituted alkylene group, —CN, —NO₂, non-substituted or substituted alkoxy group, —F, —Br, —Cl, —CF₃, —CO₂R⁵ (R⁵ represents a non-substituted or substituted alkyl group), —≡—R⁶ (R⁶ represents a hydrogen atom or non-substituted or substituted alkyl group) or 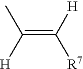
where R⁷ represents a hydrogen atom, or substituted or non-substituted alkyl group;
and at least one polymerizable compound represented by formula (2), (3) or (4) below: 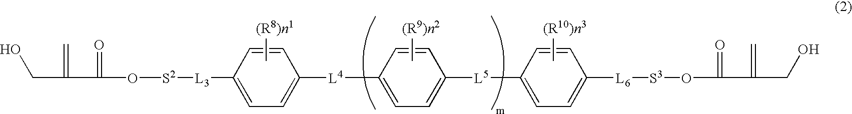
where each of R⁸, R⁹ and R¹⁰ independently represents —F, —Br, —Cl, —CH₃ or —OCH₃ and is same or different at each occurrence; each of n¹ to n³ independently represents an integer of 0 to 4; each of S² and S³ independently represents a divalent linking group selected from the group consisting of a single bond, —O—, —S—, —C(═O)—, —SO₂—, —NH—, —CH₂—, —CH═CH— and —C≡C—, and combinations of them, wherein any of which containing hydrogen atom(s) may have substituent(s) in place of the hydrogen atom(s); each of L³ and L⁶ independently represents a single bond, —O—, —S—, —NR¹¹— (R¹¹ represents a hydrogen atom of methyl group), —CO₂—, —OCO₂— or —OCO—; each of L⁴ and L⁵ independently represents a single bond, —CO₂—, —OCO—, —CONR¹²— (R¹² represents a hydrogen atom or methyl group), —NR¹³CO— (R¹³ represents a hydrogen atom or methyl group), —O—, —S—, —C(CH₃)₂—, —≡— or 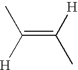
where m represents an integer of 0 to 3; 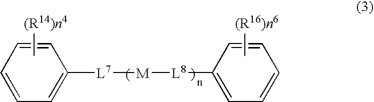
where each of R¹⁴ and R¹⁶ independently represents Q-L⁹-S⁴-L¹⁰, where Q represents a hydrogen atom or polymerizable group, each of L⁹ and L¹⁰ independently represents a single bond, —O—, —S—, —NR¹⁷— (R¹⁷ represents a hydrogen atom or methyl group), —CO₂—, —OCO₂— or —OCO—, and S⁴ represents a divalent linking group selected from the group consisting of a single bond, —O—, —S—, —C(═O)—, —SO₂—, —NH—, —CH₂—, —CH═CH—, —C≡C— and combinations of them, wherein any of which having hydrogen atom(s) may have substituent(s) in place of the hydrogen atom(s); each of n⁴ and n⁶ independently represents an integer of 0 to 5 and the sum of n⁴ and n⁶ is from 1 to 10, n⁴ “R¹⁴” s and n⁶ “R¹⁶”s may independently differ from each other, at least one of n⁴ “R¹⁴”s and n⁶ “R¹⁶”s has a polymerizable group Q; M represents the group shown below: 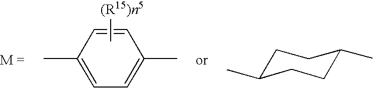
where R¹⁵ represents —F, —Br, —Cl, —CH₃ or —OCH₃, n⁵ represents an integer of 0 to 4, and n⁵ “R¹⁵”s may differ from each other; each of L⁷ and L⁸ independently represents a single bond, —CO₂—, —OCO—, —CONR¹⁷— (R¹⁷ represents a hydrogen atom or methyl group), —NR¹⁸CO— (R¹⁸ represents a hydrogen atom or methyl group), —O—, —S—, —C(CH₃)₂—, —≡— or 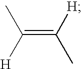
and n represents an integer from 0 to 3; provided that formula (3) has at least one polymerizable group; 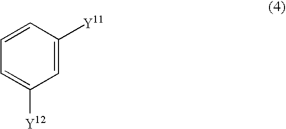
where each of Y¹¹ and Y¹² independently represents formula (4-A), formula (4-B) or formula (4-C) below, provided that formula (4) has at least one polymerizable group: 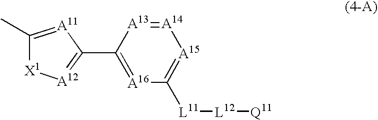
where A¹¹, A¹², A¹³, A¹⁴, A¹⁵ and A¹⁶ each represent a methine or nitrogen atom; X¹ represents an oxygen atom, sulfur atom, methylene or imino; L¹¹ represents —O—, —O—CO—, —CO—O—, —O—CO—O—, —S—, —NH—, —SO₂—, —CH₂—, —CH═CH— or —C≡C—; L¹² represents a divalent linking group selected from the group consisting of —O—, —S—, —C(═O)—, —SO₂—, —NH—, —CH₂—, —CH═CH—, —C≡C— and combinations thereof wherein any of which having hydrogen atom(s) may have substituent(s) in place of the hydrogen atom(s); and Q¹¹ represents a polymerizable group or hydrogen atom; 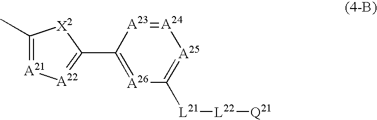
where A²¹, A²², A²³, A²⁴, A²⁵ and A²⁶ each independently represent a methine or nitrogen atom; X² represents an oxygen atom, sulfur atom, methylene or imino; L²¹ represents —O—, —O—CO—, —CO—O—, —O—CO—O—, —S—, —NH—, —SO₂—, —CH₂—, —CH═CH— or —C≡C—; L²² represents a divalent linking group selected from the group consisting of —O—, —S—, —C(═O)—, —SO₂, —NH—, —CH₂—, —CH═CH—, —C≡C— and combinations thereof wherein any of which having hydrogen atom(s) may have substituent(s) in place of the hydrogen atom(s); and Q²¹ represents a polymerizable group or hydrogen atom; 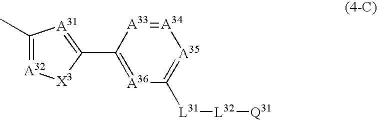
where A³¹, A³², A³³, A³⁴, A³⁵ and A³⁶ each independently represents a methine or nitrogen atom; X³ represents an oxygen atom, sulfur atom, methylene or imino; L³¹ represents —O—, —O—CO—, —CO—O—, —O—CO—O—, —S—, —NH—, —SO₂—, —CH₂—, —CH═CH— or —C≡C—; L³² represents a divalent linking group selected from the group consisting of —O—, —S—, —C(═O)—, —SO₂—, —NH—, —CH₂—, —CH═CH—, —C≡C— and combinations thereof wherein any of which having hydrogen atom(s) may have substituent(s) in place of the hydrogen atom(s); and Q³¹ represents a polymerizable group or hydrogen atom.

7. The composition for retardation film of claim 6, further comprising a polymerization initiator.

8. A photo-alignment film formed of a composition as set forth in claim 1.

9. A photo-alignment film formed of a composition as set forth in claim 1 irradiated with light.

10. A retardation film formed of a composition as set forth in claim 6.

11. A retardation film formed of a composition as set forth in claim 6 irradiated with light.

12. A liquid crystal cell comprising a pair of substrates and a liquid crystal composition held therebetween, wherein at least one of said pair of substrates has a photo-alignment film as set forth in claim 8 on the inner surface thereof.

13. A liquid crystal display device comprising a liquid crystal cell as set forth in claim 12.

14. The liquid crystal display device of claim 13, employing an IPS-mode or TN-mode.

15. A liquid crystal display device comprising a retardation film as set forth in claim 10.

16. A method of producing a photo-alignment film comprising applying a composition as set forth in claim 1 to a surface to form a layer of the composition, and irradiating the layer with polarized light or irradiating with non-polarized light in an oblique direction.

17. A method of producing a retardation film comprising applying a composition as set forth in claim 6 to a surface to form a layer of the composition, and irradiating the layer with polarized light or irradiating with non-polarized light in an oblique direction.

18. The method of claim 16, further comprising heating the layer of the composition following the irradiating.

19. The method of claim 17, further comprising heating the layer of the composition following the irradiating.

20. The method of claim 18, wherein the heating is carried out at 50° C. to 240° C.

21. The method of claim 19, wherein the heating is carried out at 50° C. to 240° C.

22. The method of claim 16, further comprising irradiating the layer with non-polarized light in normal line direction relative to the layer plane, following the irradiating with polarized light or irradiating with non-polarized light in an oblique direction.

23. The method of claim 17, further comprising irradiating the layer with non-polarized light in normal line direction relative to the layer plane, following the irradiating with polarized light or irradiating with non-polarized light in an oblique direction.

24. A liquid crystal compound represented by formula (2): 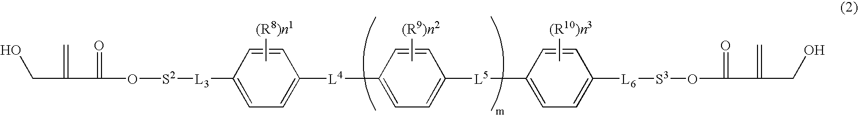
where each of R⁸, R⁹ and R¹⁰ independently represents —F, —Br, —Cl, —CH₃ or —OCH₃ and is same or different at each occurrence; each of n¹ to n³ independently represents an integer of 0 to 4; each of S² and S³ independently represents a divalent linking group selected from the group consisting of a single bond, —O—, —S—, —C(═O)—, —SO₂—, —NH—, —CH₂—, —CH═CH— and —C≡C—, and combinations of them, wherein any of which containing hydrogen atom(s) may have substituent(s) in place of the hydrogen atom(s); each of L³ and independently represents a single bond, —O—, —S—, —NR¹¹— (R¹¹ represents a hydrogen atom of methyl group), —CO₂—, —OCO₂— or —OCO—; each of L⁴ and L⁵ independently represents a single bond, —CO₂—, —OCO—, —CONR¹²— (R¹² represents a hydrogen atom or methyl group), —NR¹³CO— (R¹³ represents a hydrogen atom or methyl group), —O—, —S—, —C(CH₃)₂—, —≡— or 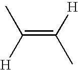
where m represents an integer of 0 to 3.
